SEQUENCE LISTING (45 sequences)

SEQ ID NO: 1 - protein, 447 aa, Artificial Sequence
  Synthetic Sequence_XBR1-402 HC amino acid sequence
QEQQKESGGGLFKPTDTLTLTCTASGFDISSYYMSWVRQAPGNGLEWIGAIGISGNAYYASWAKSRSTITRNTNLNTVTLKMTSLTAADTATYFCARDHPTYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 212 aa, Artificial Sequence
  Synthetic Sequence_XBR1-402 LC amino acid sequence
SYELTQLPSVSVSLGQTARITCEGNNIGSKAVHWYQQKPGLAPGLLIYDDDERPSGVPDRFSGSNSGDTATLTISGAQAGDEADYYCQVWDSSAYVFGGGTQLTVTGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 3 - protein, 447 aa, Artificial Sequence
  Synthetic Sequence_Ac10 HC amino acid sequence
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 218 aa, Artificial Sequence
  Synthetic Sequence_Ac10 LC amino acid sequence
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 448 aa, Artificial Sequence
  Synthetic Sequence_2A2 HC amino acid sequence
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  Synthetic Sequence_2A2 LC amino acid sequence
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 5 aa, Artificial Sequence
  Synthetic Sequence_Staphylococcus aureus sortase A recognition sequence 1, with X being any amino acid
  Xaa (3)..(3): Xaa can be any amino acid
LPXTG

SEQ ID NO: 8 - protein, 5 aa, Artificial Sequence
  Synthetic Sequence_Staphylococcus aureus sortase A recognition sequence 2, with X being any amino acid
  Xaa (3)..(3): Xaa can be any amino acid
LPXAG

SEQ ID NO: 9 - protein, 5 aa, Artificial Sequence
  Synthetic Sequence_recognition sequence for Staphylococcus aureus sortase A or engineered sortase A 4S-9 from Staphylococcus aureu
  Xaa (3)..(3): Xaa can be any amino acid
LPXSG

SEQ ID NO: 10 - protein, 5 aa, Artificial Sequence
  Synthetic Sequence_recognition sequence for engineered sortase A 2A-9 from Staphylococcus aureus, with X being any amino acid
  Xaa (3)..(3): Xaa can be any amino acid
LAXTG

SEQ ID NO: 11 - protein, 5 aa, Artificial Sequence
  Synthetic Sequence_Streptococcus pyogenes sortase A recognition sequence, with X being any amino acid
  Xaa (3)..(3): Xaa can be any amino acid
LPXTA

SEQ ID NO: 12 - protein, 5 aa, Artificial Sequence
  Synthetic Sequence_Staphylococcus aureus sortase recognition sequence
NPQTN

SEQ ID NO: 13 - protein, 5 aa, Artificial Sequence
  Synthetic Sequence_Linker derived from Staphylococcus aureus sortase A recognition sequence 1, with X being any amino acid and n ? 1 and ? 21
  Xaa (3)..(3): Xaa can be any amino acid
LPXTG

SEQ ID NO: 14 - protein, 5 aa, Artificial Sequence
  Synthetic Sequence_Linker derived from Staphylococcus aureus sortase A recognition sequence 2, with X being any amino acid and n ? 1 and ? 21
  Xaa (3)..(3): Xaa can be any amino acid
LPXAG

SEQ ID NO: 15 - protein, 5 aa, Artificial Sequence
  Synthetic Sequence_Linker derived from recognition sequence for Staphylococcus aureus sortase A or engineered sortase A 4S-9 from Staphylococcus aureus, with X being any amino acid and n ? 1 and ? 21
  Xaa (3)..(3): Xaa can be any amino acid
LPXSG

SEQ ID NO: 16 - protein, 5 aa, Artificial Sequence
  Synthetic Sequence_Linker derived from recognition sequence for engineered sortase A 2A-9 from Staphylococcus aureus, with X being any amino acid and n ? 1 and ? 21
  Xaa (3)..(3): Xaa can be any amino acid
LAXTG

SEQ ID NO: 17 - protein, 5 aa, Artificial Sequence
  Synthetic Sequence_Linker derived from Streptococcus pyogenes sortase A recognition sequence, with X being any amino acid and n ? 1 and ? 21
  Xaa (3)..(3): Xaa can be any amino acid
  Xaa (5)..(5): Xaa is G or A
LPXTX

SEQ ID NO: 18 - protein, 5 aa, Artificial Sequence
  Synthetic Sequence_Linker derived from Staphylococcus aureus sortase recognition sequence, with n ? 1 and ? 21
NPQTG

SEQ ID NO: 19 - protein, 19 aa, Artificial Sequence
  Synthetic Sequence_leader sequence
MNFGLRLIFLVLTLKGVQC

SEQ ID NO: 20 - protein, 447 aa, Artificial Sequence
  Synthetic Sequence_huXBR1-402-17 HC amino acid sequence
QVQLRESGPGLVKPSETLSLTCTVSGFDISSYYMSWVRQPPGKGLEWIGAIGISGNAYYASWAKSRVTISRDTSKNQFSLKLSSVTAADTAVYYCARDHPTYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 212 aa, Artificial Sequence
  Synthetic Sequence_huXBR1-402-17 LC amino acid sequence
SYELTQPPSVSVAPGKTARITCEGNNIGSKAVHWYQQKPGQAPVLVIYDDDERPSGIPERFSGSNSGNTATLTISRVEAGDEADYYCQVWDSSAYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - protein, 5 aa, Artificial Sequence
  Synthetic Sequence_XBR1-402 HCDR1
SYYMS

SEQ ID NO: 23 - protein, 16 aa, Artificial Sequence
  Synthetic Sequence_XBR1-402 HCDR2
AIGISGNAYYASWAKS

SEQ ID NO: 24 - protein, 9 aa, Artificial Sequence
  Synthetic Sequence_XBR1-402 HCDR3
DHPTYGMDL

SEQ ID NO: 25 - protein, 11 aa, Artificial Sequence
  Synthetic Sequence_XBR1-402 LCDR1
EGNNIGSKAVH

SEQ ID NO: 26 - protein, 7 aa, Artificial Sequence
  Synthetic Sequence_XBR1-402 LCDR2
DDDERPS

SEQ ID NO: 27 - protein, 9 aa, Artificial Sequence
  Synthetic Sequence_XBR1-402 LCDR3
QVWDSSAYV

SEQ ID NO: 28 - protein, 9 aa, Artificial Sequence
  Synthetic Sequence_Ac10 HCDR1
YTFTDYYIT

SEQ ID NO: 29 - protein, 14 aa, Artificial Sequence
  Synthetic Sequence_Ac10 HCDR2
WIGWIYPGSGNTKY

SEQ ID NO: 30 - protein, 9 aa, Artificial Sequence
  Synthetic Sequence_Ac10 HCDR3
NYGNYWFAY

SEQ ID NO: 31 - protein, 12 aa, Artificial Sequence
  Synthetic Sequence_Ac10 LCDR1
QSVDFDGDSYMN

SEQ ID NO: 32 - protein, 11 aa, Artificial Sequence
  Synthetic Sequence_Ac10 LCDR2
VLIYAASNLES

SEQ ID NO: 33 - protein, 8 aa, Artificial Sequence
  Synthetic Sequence_Ac10 LCDR3
QQSNEDPW

SEQ ID NO: 34 - protein, 9 aa, Artificial Sequence
  Synthetic Sequence_2A2 HCDR1
YTFSDYEMH

SEQ ID NO: 35 - protein, 14 aa, Artificial Sequence
  Synthetic Sequence_2A2 HCDR2
WIGAIDPETGGTAY

SEQ ID NO: 36 - protein, 10 aa, Artificial Sequence
  Synthetic Sequence_2A2 HCDR3
GYYDYDSFTY

SEQ ID NO: 37 - protein, 8 aa, Artificial Sequence
  Synthetic Sequence_2A2 LCDR1
QNVDAAVA

SEQ ID NO: 38 - protein, 11 aa, Artificial Sequence
  Synthetic Sequence_2A2 LCDR2
LLIYSASNRYT

SEQ ID NO: 39 - protein, 8 aa, Artificial Sequence
  Synthetic Sequence_2A2 LCDR3
QQYDIYPY

SEQ ID NO: 40 - protein, 9 aa, Artificial Sequence
  Synthetic Sequence_huXBR1-402-17 HCDR1
FDISSYYMS

SEQ ID NO: 41 - protein, 17 aa, Artificial Sequence
  Synthetic Sequence_huXBR1-402-17 HCDR2
WIGAIGISGNAYYASWA

SEQ ID NO: 42 - protein, 10 aa, Artificial Sequence
  Synthetic Sequence_huXBR1-402-17 HCDR3
RDHPTYGMDL

SEQ ID NO: 43 - protein, 8 aa, Artificial Sequence
  Synthetic Sequence_huXBR1-402-17 LCDR1
NIGSKAVH

SEQ ID NO: 44 - protein, 11 aa, Artificial Sequence
  Synthetic Sequence_huXBR1-402-17 LCDR2
LVIYDDDERPS

SEQ ID NO: 45 - protein, 8 aa, Artificial Sequence
  Synthetic Sequence_huXBR1-402-17 LCDR3
QVWDSSAY

(19) United States
(12) Patent Application Publication
(10) Pub. No.: US 2021/0379194 A1
(43) Pub. Date: Dec. 9, 2021

(54) BINDING PROTEIN-TOXIN CONJUGATES COMPRISING ANTHRACYCLINES, AND USE THEREOF IN IMMUNE-ONCOLOGICAL APPLICATIONS

(71) Applicant: NBE-Therapeutics AG, Basel (CH)

(72) Inventors: Ulf GRAWUNDER, Hersberg (CH); Roger BEERLI, Adlikon bei Regensdorf (CH); Lorenz WALDMEIER, Basel (CH); Francesca PRETTO, Schaffhausen (CH)

(21) Appl. No.: 17/283,924

(22) PCT Filed: Oct. 11, 2019
(86) PCT No.: PCT/EP2019/077644

§ 371 Date: Apr. 8, 2021

(30) Foreign Application Priority Data
Oct. 11, 2018 (EP) 18199879.0

(51) Int. Cl. A61K 47/68; A61P 35/00

(52) U.S. Cl. CPC A61K 47/6809; A61P 35/00; A61K 47/6889; A61K 47/6851

(57) ABSTRACT

The present invention relates to binding protein-toxin conjugates comprising one or more anthracycline toxin moieties, and the use thereof in immunooncological applications.

### FIELD OF THE INVENTION

The present invention relates to binding protein-toxin conjugates comprising one or more anthracycline toxin moieties, and the use thereof in immune-oncological applications.

### BACKGROUND

Binding protein-toxin conjugates, with antibody-drug-conjugates (ADC) as their most prominent representatives, have had a tremendous impact in cancer therapy, providing new therapeutic approaches for tumor types, which so far could not be treated.

On the other hand, the discovery of immune checkpoints, and the development of immune checkpoint inhibitors, has again brought into focus the role of the immune system in cancer therapy, and ways to stimulate the immune system, or to overcome immune checkpoints that the respective tumors use for self protection against immune system attacks.

Although both approaches have been discussed very enthusiastically, and have demonstrated significant progress in the treatment of cancer, it also became clear that there are tumor types which cannot be treated with these new approaches.

It has for example been shown that ADCs with particular toxins are not active against specific cancer types, although they address, by their antibody, a cancer specific antigen. It has also been shown that tumor types exist where immune checkpoint inhibitors have no efficacy.

It is hence one object of the present invention to provide new approaches which make tumor types treatable which could so far not be addressed by existing therapies.

It is one further object of the present invention to provide combination therapies which increase the anti-tumor activity of the corresponding mono therapies.

These and further objects are met with methods and means according to the independent claims of the present invention. The dependent claims are related to specific embodiments.

### SUMMARY OF THE INVENTION

The present invention provides binding protein-toxin conjugates comprising one or more anthracycline toxin moieties, and the use thereof in immune-oncological applications. The invention and general advantages of its features will be discussed in detail below.

### DESCRIPTION OF THE FIGURES

FIG. 1. Chemical structures of pentaglycine-modified EDA-anthracycline PNU-159682 derivative (G5-EDA-PNU or G5-PNU) used for the generation of site-specifically conjugated ADCs used in the experiments in this invention. G3-PNU and G2-PNU refer to the same structures but wherein the pentaglycine is replaced by a triglycine or a diglycine, respectively.

FIG. 2. FACS analysis for expression of human ROR1 stably expressed on mouse EMT-6 breast cancer cells. EMT-6-ROR1 clone 14 selected for in vivo studies has been analyzed by FACS staining for ROR1 expression with the fluorescently labeled anti-ROR1 antibody clone 2A2. The negative control shows a staining of the same cells with a fluorescently labeled isotype-matched control antibody.

FIG. 3. Dose titration of novel anti-ROR1 PNU-ADC, based on antibody XBR1-402, with a single treatment at different dose levels (0.25, 0.5 and 1 mg/kg) in orthotopic syngeneic breast cancer model EMT-6-ROR1 (clone 14). (A) Tumor growth of the orthotopically transplanted breast tumors is monitored in the different treatment groups over time as indicated. (B) tumor growth curves in individual mice of the groups displayed in panel (A).

FIG. 4. Tumor volume evolution in orthotopically transplanted syngeneic mice with EMT-6-ROR1 (clone 14) breast cancer cells, either (A) with injection of vehicle control (PBS), or with (B) single administration of 0.25 mg/kg with an isotype-matched control PNU-ADC comprising anti-CD30 antibody brentuximab, clone Ac10, or (C) single administration of 0.25 mg/kg with anti-ROR1 PNU-ADC comprising novel anti-ROR1 antibody XBR1-402, which according to FIG. 3 has been determined to be a suboptimal dose for treatment of orthotopic EMT-6-ROR1 breast tumors in mice. Panels (D) and (E) show combination treatments with anti-CTLA4 immune-checkpoint inhibitor antibody 9D9 with 0.25 mg/kg of isotype-matched control ADC Ac10 and 0.25 mg/kg novel anti-ROR1 PNU ADC, as indicated. (F) Median group tumor volume evolution (with application of Last-Observation-Carried-Forward (LOCF) methodology) for the following groups: vehicle control, Ac10-G5-PNU isotype control and XBR1-402-G5-PNU, (G) Median group tumor volume evolution (with application of Last-Observation-Carried-Forward (LOCF) methodology) for the following groups: vehicle control, Ac10-G5-PNU isotype control and anti-mouse CTLA-4 antibody 9D9, and XBR1-402-G5-PNU and anti-mouse CTLA-4 antibody 9D9.

FIG. 5. FACS analysis for expression of human ROR1 stably expressed on (A) mouse CT-26 breast cancer cells (clone 3) and (B) B16 mouse melanoma cancer cells (pool). CT-26-ROR1 clone 3 and B16-ROR1 pools selected for in vivo studies have been analyzed by FACS staining for ROR1 expression with the fluorescently labeled anti-ROR1 antibody clone XBR1-402 (right peak). The negative control shows a staining of the same cells with a fluorescently labeled isotype-matched control antibody (left peak).

FIG. 6. Tumor volume evolution in mice transplanted with CT-26-ROR1 clone 3 tumor cells as per Example 6: (A) vehicle control (untreated) of group 1, (B) isotype control of group 2, (C) anti-ROR1 ADC huXBR1-402-17 of group 3, (D) isotype control plus immune checkpoint inhibitor anti-mouse PD-1 antibody of group 4, (E) anti-ROR1 ADC huXBR1-402-17 plus immune checkpoint inhibitor anti-mouse PD-1 antibody of group 7. The insets in panels A-E provide the number of mice in which tumor growth was prevented relative to number of mice in each treatment group. The results suggest a cooperation between the evaluated immune checkpoint inhibitor and the targeted PNU derivative-comprising ADC. FIG. 7. Lung surface colony volumes in mice following i.v. application of ROR1-overexpressing B16 pools, as per Example 7, following treatment with vehicle control (“vehicle”, group 1), immune checkpoint inhibitor anti-mouse CTLA-4 antibody (“CTLA-4”, group 2), anti-ROR1 ADC huXBR1-402-17 (“ADC”, group 3), and anti-ROR1 ADC huXBR1-402-17 plus immune checkpoint inhibitor anti-mouse CTLA-4 antibody (“ADC+CTLA-4”, group 4). These results suggest, in a poorly immunogenic tumor, a strong synergy between the evaluated immune checkpoint inhibitor and the targeted PNU derivative-comprising ADC. “n.s.” means not statistically significantly different, “*” indicates statistically significantly different.

FIG. 8. CD4 and CD8-staining of hROR1 (human ROR1)-overexpressing B16 tumor cryo-sections from (A) an untreated control mouse, and (B) the three mice treated with ADCs (once, at 4 mg/kg) as per the invention. As per FIG. 8(A), B16 tumors naturally contain low levels of CD4+ and CD8+ cells, in keeping with the status of B16 tumors as cold tumors.

FIG. 8(B) shows that treatment with the ADC, as per the present invention, increases the number of CD4+ and CD8+ cells within the tumor.

FIG. 9. Analysis of 41BB (CD137) staining, a marker of T cells activation, on infiltrating lymphocytes in hROR1 (human ROR1)-overexpressing CT-26 and B-16 tumors. As per FIG. 9, a significantly lower number of activated T CD4+(A) and CD8+(B) lymphocytes can be observed within B-16 tumors in comparison to CT-26 tumors, in keeping with the status of B-16 tumors as cold tumors. “*” indicates a statistically significantly difference.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, to prior art documents that disclose standard or routine methods. In that case, the incorporation by reference has mainly the purpose to provide sufficient enabling disclosure, and avoid lengthy repetitions.

According to one aspect of the invention, a binding protein-toxin conjugate is provided comprising one or more anthracycline toxin moieties conjugated to a binding protein (for the manufacture of a medicament) for the treatment of a patient
suffering from,
being at risk of developing, and/or
being diagnosed with

a neoplastic disease characterized as being a cold tumor.

It is important to emphasize that the above language, with the term “for the manufacture of a medicament” in brackets, has been chosen to comply within different national/regional requirements as to claims covering medical uses of given compounds. The language shall in particular encompass both the so-called swiss type format (where the qualifier “for the manufacture of a medicament” is needed) as well as the respective claim language under EPC2000 (where the same qualifier is no longer admissible).

According to another aspect of the invention, a method of treating or preventing a neoplastic disease characterized as being a cold tumor is provided, said method comprising administering to a patient a binding protein-toxin conjugate comprising one or more anthracycline toxin moieties.

According to another aspect of the invention, a combination of
(i) a binding protein-toxin conjugate comprising one or more anthracycline toxin moieties, conjugated to a binding protein, and
(ii) an immune checkpoint inhibitor

is provided (for the manufacture of a medicament) for the treatment of a patient
suffering from,
being at risk of developing, and/or
being diagnosed with

a neoplastic disease characterized as being a cold tumor,

wherein the binding protein-toxin conjugate and the immune checkpoint inhibitor are administered to the patient simultaneously or sequentially, in any order.

According to another aspect of the invention, a method of treating or preventing a neoplastic disease characterized as being a cold tumor is provided, said method comprising administering to a patient
(i) a binding protein-toxin conjugate comprising one or more anthracycline toxin moieties, conjugated to a binding protein, and
(ii) an immune checkpoint inhibitor

wherein the binding protein-toxin conjugate and the immune checkpoint inhibitor are administered to the patient simultaneously or sequentially, in any order.

According to another aspect of the invention, a binding protein-toxin conjugate comprising one or more anthracycline toxin moieties, conjugated to a binding protein, is provided for use in combination with an immune checkpoint inhibitor (for the manufacture of a medicament) for use in the treatment of a patient
suffering from,
being at risk of developing, and/or
being diagnosed with

a neoplastic disease characterized as being a cold tumor.

According to another aspect of the invention, an immune checkpoint inhibitor for use in combination with a binding protein-toxin conjugate comprising one or more anthracycline toxin moieties, conjugated to a binding protein, (for the manufacture of a medicament) for use in the treatment of a patient
suffering from,
being at risk of developing, and/or
being diagnosed with

a neoplastic disease characterized as being a cold tumor.

In one embodiment, the immune checkpoint inhibitor modulates a positive immune checkpoint protein. In another embodiment, the immune checkpoint inhibitor modulates a negative immune checkpoint protein.

In one embodiment, the binding protein-toxin conjugate comprising one or more anthracycline toxin is administered before the immune checkpoint inhibitor.

In one embodiment, the patient
is suffering from, and/or
diagnosed with

a neoplastic disease characterized as being a cold tumor.

A “hot tumor” defines a cancerous or neoplastic tissue which is characterized by immune cell infiltration. The level of immune infiltration reflects whether the immune system is recognizing and engaging the tumor. The term “hot tumor” is synonymously used with the term “immunologically responsive tumor” herein. Alternatively, “hot tumors” may be defined as tumors that respond to treatment by immune checkpoint inhibitors.

As used herein, the term “respond to treatment” means a statistically significant reduction of tumor burden or growth relative to a comparable vehicle or isotype control treatment.

In one embodiment, the term “cold tumor” defines a cancerous or neoplastic tissue which is characterized by poor or lacking immune cell infiltration. The term “cold tumor” is synonymously used with the term “immunologically unresponsive tumor” herein.

One benchmark for a tumor to qualify as cold is the so-called Immunoscore, based on the density of two types of T cells in a sample of the tumor. One example is the density of two lymphocyte populations (CD3/CD45RO, CD3/CD8 or CD8/CD45RO), both in the core of the tumour (CT) and in the invasive margin (IM) of tumors. The Immunoscore provides a score ranging from Immunoscore 0 (I0) when low densities of both cell types are found in both regions, to Immunoscore 4 (I4), when high densities are found in both regions.

Methods to determine the Immunoscore have been described, e.g., in Galon et al. 2014, Pages et al. 2009, Angell et al. 2013, and Galon et al. 2013, the contents of which are incorporated by reference herein.

So far, it has been discussed that tumors with a low Immunoscore (“cold tumors”) are not very responsive to treatment with immune checkpoint inhibitors, like anti PD-1, anti CTLA-4 or anti-PD-L1 antibodies, while tumors with a high Immunoscore are responsive to such treatment (see Bu et al. 2016). Without being bound to theory, the rationale behind this finding could be that, if there is no immune engagement in a respective tumor, inhibition of an immune checkpoint by a given inhibitor cannot change anything to the better.

The inventors realized that a binding protein-toxin conjugate comprising one or more anthracycline toxin moieties has particular immunologic efficacy in tumors, resulting, inter alia, in an immune protection from tumor re-challenge. This surprising finding could not be anticipated from the findings made with immune checkpoint inhibitors, like anti-PD-1, anti-CTLA-4 or anti-PD-L1 antibodies. The rationale that applies for the latter does not likely apply to binding protein-toxin conjugate comprising one or more anthracycline toxin moieties.

According to one embodiment of the invention, the neoplastic disease characterized as being a cold tumor is a tumor that is or has been refractory, resistant to immune checkpoint inhibitor treatment, or recurrent after immune checkpoint inhibitor treatment.

As used herein, the term “refractory” refers to a tumor that fails, or has failed, to respond reasonably in a favorable manner in terms of tumor shrinkage or duration of stabilization or shrinkage in response to treatment with an immune checkpoint inhibitor.

As used herein, the term “resistant” refers to a tumor that fails, or has failed, to respond reasonably in a favorable manner in terms of tumor shrinkage or duration of stabilization or shrinkage in response to treatment with an immune checkpoint inhibitor for a time of greater than 3 months or more.

As used herein, the term “recurrent” refers to a tumor that hat has come back after treatment with an immune checkpoint inhibitor, usually after a period of time during which the tumor could not be detected. The tumor may come back to the same place or to another place in the body of a subject.

Such treatment with an immune checkpoint inhibitor has preferably been a monotheraopy.

Such treatment with an immune checkpoint inhibitor has preferably been administered in a dosage or regiment recommended in the respective drug label or SOPC.

According to one embodiment of the invention, the neoplastic disease characterized as being a cold tumor is selected from the group consisting of
melanoma,
colorectal cancers or tumors,
pancreatic cancers or tumors
glioblastoma,
ovarian cancers or tumors, and/or
prostrate cancers or tumors.

According to one embodiment of the invention, the binding protein binds to at least one target selected from the group consisting of
ROR1
CS1
HER2
Mesothelin (MN), and/or
ROR2.

Among these, ROR1 is a preferred target.

These targets, and antibodies theregainst, are described in the following publications
US2019112385A1 (Mesothelin)
US2019153092A1, WO2019016381 (ROR1)
WO2019030240A1 (CS-1),
US2018028682 (HER2) and
WO2019016392A1 (ROR2), the content of which is incorporated by reference herein.

Preferably, the said targets are of human origin. In particular, ROR1, when mentioned herein, means, preferably, human ROR1.

As used herein, the term “immune checkpoint inhibitor” refers to any binding agent or compound suitable to act against an immune-checkpoint protein. In particular, an “immune checkpoint inhibitor” refers to any binding agent or compound suitable to modulate the activity of an immune-checkpoint protein in order to support activity of the immune system and notably the ability of cells to recognize cancer cells and act against them. Immune checkpoint proteins may be positive (i.e., they support T cell activity and an immune response) or negative (i.e., they limit d cell activity and an immune response). In the case of a positive immune checkpoint protein, an immune checkpoint inhibitor means a binding agent or compound suitable to promote the activity of this immune checkpoint protein. In the case of a negative immune checkpoint protein, an immune checkpoint inhibitor means a binding agent or compound suitable to inhibit the activity of this immune checkpoint protein.

An immune checkpoint is a protein that regulates the immune system (Pardoll D. M. “The blockage of immune checkpoints in cancer immunotherapy”, Nature, 2012, Volume 12, p. 252-264), and is crucial for self-tolerance, which prevents the immune system from attacking cells indiscriminately, preferably selected from the group as disclosed in the following table:

| | | | | Positive or |
|---|---|---|---|---|
| | | | | Negative |
| | | | | immune |
| | | | | checkpoint |
| Type | Alias | Full name | Uni Prot | protein |
| PD-1 | CD279 | Programmed cell death protein 1 | Q15116 | Negative |
| CTLA-4 | CD152 | Cytotoxic T-lymphocyte-associated | P16410 | Negative |
| | | Protein 4 | | |
| PD-L1 | CD274, B7 | Programmed cell death ligand 1 | Q9NZQ7 | Negative |
| | Homolog 1 | | | |
| PD-L2 | B7-DC, | Programmed cell death ligand 2 | Q9BQ51 | Negative |
| | CD273 | | | |
| LAG3 | CD223 | Lymphocyte-activation gene 3 | P18627 | Negative |
| CD40 | TNFRSF5 | Tumor necrosis factor receptor | P25942 | Positive |
| | | superfamily member 5 | | |
| CD40L | CD154 | CD154 Ligand | P29965 | Positive |
| TIM3 | HAVCR2 | Hepatitis A virus cellular receptor 2 | Q8TDQ0 | Negative |
| OX40 | TNFRSF4, | Tumor necrosis factor receptor | P43489 | Positive |
| | CD134 | superfamily, member 4 | | |
| OX40L | | ligand for CD134L | P23510 | Positive |
| CD112 | PVRL2, | Poliovirus receptor-related 2 | Q92692 | |
| | nectin-2 | | | |
| CD155 | | poliovirus receptor | P15151 | Negative |
| B7-H3 | CD276 | | Q5ZPR3 | Negative |
| B7-H4 | VTCN1 | V-set domain-containing T-cell | Q7Z7D3 | Positive |
| | | activation inhibitor 1 | | |
| IDO1 | | Indolamin-2,3-Dioxygenase 1 | P14902 | |
| IDO2 | | Indolamin-2,3-Dioxygenase 2 | Q6ZQW0 | |
| TDO2 | | Tryptophan 2,3-dioxygenase | P48775 | |
| TIGIT | WUCAM | T cell immunoreceptor with Ig and | Q495A1 | Negative |
| | Vstm3 | ITIM domains | | |
| Galectin-9 | | | O00182 | Negative |
| GITR | | Glucocorticoid-induced tumor necrosis | Q9Y5U5 | Positive |
| | | factor receptor | | |
| GITRL | | Glucocorticoid-induced tumor necrosis | Q9UNG2 | Positive |
| | | factor receptor ligand | | |

For example, the PD-1 checkpoint protein expressed on the surface of T cells acts to prevent those T cells from attacking other cells in the body. When T cell PD-1 binds to PD-L1 on the surface of a cell, the T cell will not trigger an immune response against that cell. Certain cancer cells express high levels of PD-L1 which helps them avoid immune system attack. Accordingly, antibodies that bind either PD-1 or PD-L1 and according inhibit their binding one another serve to promote an immune response against cancer cells. CTLA-4 is another such negative immune checkpoint protein expressed on T cells.

According to a one group of embodiments, the neoplastic disease is characterized as being a cold tumor characterized as having an immunoscore of I<1.

The Immunoscore is a method to estimate the prognosis of cancer patients, based on the immune cells that infiltrate cancer and surround it. The Immunoscore has been internationally validated in colorectal cancer. It is based Galon et al's (2006) findings who revealed a positive association of cytotoxic and memory T cells with survival of colorectal cancer patients.

Immunoscore incorporates the effects of the host immune response into cancer classification and improves prognostic accuracy. It measures the density of two T lymphocyte populations (CD3/CD8, CD3/CD45RO or CD8/CD45RO) in the center and at the periphery of the tumor. The Immunoscore provides a score ranging from 0 (I0) when low densities of both cell types are found in both regions, to Immunoscore 4 (I4) when high densities are found in both regions. The Immunoscore does not measure how the immune cells in the periphery of the tumor are organized, or the amount of B-cells, tertiary lymphoid structures and germinal centers. All of these have important roles in the immune response to colorectal and other cancers.

In one embodiment, the cold tumor is a tumor having an immunoscore of I0 or I1, and preferably is a tumor having an immunoscore of I1.

In another embodiment, the term “cold tumor” defines a cancerous or neoplastic tissue which is characterized by a lack of, or insufficient, immune cell infiltration. The level of immune infiltration reflects whether the immune system is recognizing the tumor.

In another embodiment, a cold tumor is a tumor that does not respond to an immune checkpoint inhibitor when said immune checkpoint inhibitor is dosed in vivo as a monotherapy. In this embodiment and relative to an immunocompetent mouse model, a cold tumor is be a tumor which, once established in the mouse, does not respond significantly (relative to a vehicle control group) to an immune checkpoint inhibitor dosed as a monotherapy at up to about 10 mg/kg, or up to 20 mg/kg, or up to the maximum tolerated dose (MTD) in that mouse model. In a human, a cold tumor is, in another embodiment, a tumor that does not respond significantly to an immune checkpoint inhibitor dosed according to the immune checkpoint inhibitor label, e.g., as a monotherapy at up to 10 mg/kg, e.g., at 1 mg/kg, at 2 mg/kg, at 3 mg/kg, or at 200 or 240 mg, optionally with dosing every 2 to 3 weeks.

In another embodiment, and relative to human solid tumors, a cold tumor can be defined relative to RECIST (Response Evaluation Criteria In Solid Tumors) criteria as published in February 2000 (Therasse P. et al., “New Guidelines to Evaluate the Response to Treatment in Solid Tumors”, Journal of the National Cancer Institute, Vol. 92, No. 3, Feb. 2, 2000) and updated in 2009 (Eisenhauer E. A. et al., “New response evaluation criteria in solid tumours: Revised RECIST guideline (version 1.1)”, European Journal of Cancer, Volume 45, Issue 2, 228-247). In this context, in another embodiment, a cold tumor is a tumor that is associated with less than a partial response (“PR”; i.e., a tumor that is progressive (“PD”, progressive disease) or remains stable (“SD”, stable disease) after treatment with one or more immune checkpoint inhibitors dosed according to the immune checkpoint inhibitor label instructions. In another embodiment, a cold tumor is a tumor that is associated with less than a partial response (“PR”; i.e., a tumor that is progressive (“PD”, progressive disease) or remains stable (“SD”, stable disease) after treatment with one or more immune checkpoint inhibitors dosed over at least 6 weeks, preferably at least 8 weeks, more preferably at least 12 weeks. In another embodiment, a cold tumor is a breast cancer tumor that is progressive after anti-PD-L1 therapy.

In another embodiment, a cold tumor is a tumor that is associated with progressive disease (“PD”) after at least 6 weeks, preferably after at least 8 weeks, more preferably after at least 12 weeks of one or more immune checkpoint inhibitors dosed according to the immune checkpoint inhibitor label instructions.

In another embodiment, a cold tumor is a tumor that becomes unresponsive to one or more immune checkpoint inhibitors, i.e., tumors that previously responded to treatment with one or more immune checkpoint inhibitors but become unresponsive.

In another embodiment, a cold tumor may also be defined as a tumor that relapses (i.e., undergoes a decreased response to treatment) after initial response to one or more immune checkpoint inhibitors, where the latter are dosed according to the immune checkpoint inhibitor label instructions.

In another embodiment, a cold tumor is a tumor that has been classified as a cold tumor by clinicians. Particular, non-limitative, examples of cold tumors include colorectal cancer tumors that are Microsatellite Instability low (MSI-low; Sinicrope, F. “The Role of Microsatellite Instability Testing in Management of Colorectal Cancer”, Clinical Advances in Hematology & Oncology Volume 14, Issue 7 Jul. 2016). Such tumors are known to be non-responsive to immune checkpoint inhibitors. These tumors can be identified as cold tumors without first treating with one or more immune checkpoint inhibitors to establish non-response.

As discussed above, “cold tumors” are not very responsive to treatment with immune checkpoint inhibitors, like anti PD-1 or anti CTLA-4 antibodies. The rationale behind this phenomenon could be that, if there is no immune system engagement in a respective tumor, inhibition of an immune checkpoint by a given inhibitor cannot change anything to the better.

In particular, a subset of melanoma, colorectal cancers or tumors, pancreatic cancers or tumors, glioblastoma, ovarian cancers or tumors and prostate cancers or tumors are known to be cold tumors.

The inventors show that, with a treatment comprising (i) a binding protein-toxin conjugate comprising one or more anthracycline toxin moieties, conjugated to a binding protein, and (ii) an immune checkpoint inhibitor, such cold tumors can efficiently be treated.

This finding is surprising, and could not be anticipated from the findings made with immune checkpoint inhibitors, like anti PD-1, anti CTLA-4 or anti-PD-L1 therapies, nor with anthracycline-comprising binding protein-toxin conjugates. Without being bound to theory, it appears that the anthracycline-comprising binding protein-toxin conjugate is capable of rendering cold tumors susceptible to immune checkpoint therapy, i.e., transforming them into hot tumors, so that they can efficiently be treated with immune checkpoint inhibitors.

Preferably, the interval within which the two different agents are administered to the patient is no longer than maximally four weeks.

In one embodiment, the binding protein-toxin conjugate comprising one or more anthracycline toxins is administered before the immune checkpoint inhibitor.

According to one embodiment, the at least one anthracycline toxin moiety is PNU-159682, which is disclosed in (Quintierei et al. 2005.

According to another embodiment, the at least one anthracycline toxin moiety is a derivative of the anthracycline PNU-159682 having the following formula (i)

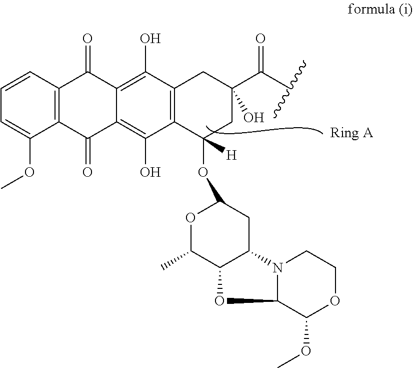

said toxin being conjugated at its wavy to the binding protein via a linker.

PNU-derived anthracyclines and the use thereof in antibody drug conjugates is enablingly disclosed in WO2016102679 assigned to the same applicant. The content of this publication is incorporated by reference herein.

According to another group of embodiments the immune checkpoint inhibitor is at least one selected from the group consisting of
anti PD-1
anti PD-L1
anti PD-L2
anti CTLA-4
anti LAG3
anti CD40 or anti CD40L
anti TIM3,
anti OX40 or anti OX40L (CD134/CD134L),
anti CD112
anti CD155
anti B7-H3
anti B7-H4
anti IDO1
anti IDO2
anti TDO2
anti TIGIT
anti GITR, and/or
anti Galectin-9.

Preferably, the checkpoint inhibitor is a compound or a monoclonal antibody antagonizing the PD-1/PD-L1 pathway implicated in the inhibition of T cell activation, more preferably a monoclonal anti-PD-1 antibody, especially nivolumab, pembrolizumab, pidilizumab or tislelizumab, or more preferably a monoclonal anti-PD-L1 antibody, more preferably atezolizumab, durvalumab, avelumab or BMS-936559.

Preferably, the checkpoint inhibitor is a compound or a monoclonal antibody antagonizing the PD-1/PD-L2 pathway implicated in the inhibition of T cell activation, more preferably a monoclonal anti-PD-1 antibody, especially nivolumab, pembrolizumab, pidilizumab or tislelizumab, or more preferably a monoclonal anti-PD-L2 antibody.

Preferably, the checkpoint inhibitor is a compound or a monoclonal antibody antagonizing the CTLA-4/B7 pathway implicated in the inhibition of T cell activation, more preferably a monoclonal anti-CTLA1-4 antibody, more preferably ipilimumab or tremelimumab.

Preferably, the checkpoint inhibitor is a compound or a monoclonal antibody antagonizing the LAG3/MHC class II pathway implicated in the inhibition of T cell stimulation and TREG activity, more preferably a monoclonal anti-LAG3 antibody, especially LAG525, BMS-986016, TSR-033, MK-4280 or REGN3767.

Preferably, the checkpoint inhibitor is a compound or a monoclonal antibody antagonizing the TIM3/Galectin-9 pathway implicated in reduced T cell function, more preferably a monoclonal anti-TIM3 antibody or a monoclonal anti-Galectin-9 antibody.

Preferably, the checkpoint inhibitor is a compound or a monoclonal antibody antagonizing the TIGIT/CD112 pathway implicated in the inhibition of T cell activation, more preferably a monoclonal anti-TIGIT antibody or a monoclonal anti-CD112 antibody.

Preferably, the checkpoint inhibitor is a compound or a monoclonal antibody antagonizing the TIGIT/CD155 pathway implicated in the inhibition of T cell activation, more preferably a monoclonal anti-TIGIT antibody or a monoclonal anti-CD155 antibody.

Preferably, the checkpoint inhibitor is a compound or a monoclonal antibody acting as an agonist on GITR, more preferably an agonist monoclonal anti-GITR antibody, especially GWN323, BMS-986156, MK-4166, MK-1248, TRX518, INCAGN1876, AMG 228, or INBRX-110.

Preferably, the checkpoint inhibitor is a compound or a monoclonal antibody acting as an agonist on OX40, especially agonist anti-OX40 monoclonal antibodies such as MOXR0916, PF-04518600, MED10562, MED16469, or MED16383.

Preferably, the checkpoint inhibitor is a compound or a monoclonal antibody acting as an agonist on CD40, especially agonist anti-CD40 monoclonal antibodies such as CP-870 or CP-893.

Among the checkpoint inhibitors discussed above, compounds or monoclonal antibodies binding or antagonizing
PD-1
PD-L1
CTLA-4, and or
B7

are particularly preferred, with PD-1 and PD-L1 being the most preferred targets.

According to another group of embodiments, the conjugate comprises at its wavy line a linker structure X-L₁-L₂-L₃-Y, wherein L₁-L₃ represent linkers, and two of L₁-L₃ are mandatory, and wherein X and Y further represent each one or more optional linkers.

According to another group of embodiments the linker structure comprises, as L₂, an oligo-glycine peptide (Gly)ₙ coupled to said anthracycline derivative, directly or by means of another linker L₁, and wherein n is an integer ≥1 and ≤21, preferably 2 to 5. It is important to understand that, in one specific embodiment (where Streptococcus pyogenes sortase A is used, see below), the oligo-glycine (Gly)ₙ can optionally be replaced by an oligo-alanine (Ala)ₙ or by mixed Gly/Ala peptides.

According to another group of embodiments, the oligo-glycine peptide (Gly)ₙ is conjugated to the anthracycline derivative of formula (i) by means of an alkylenediamino linker (EDA), designated as L₁, which alkylenediamino linker is conjugated to the anthracycline derivative by means of a first amide bond, while it is conjugated to the carboxy terminus of the oligo-glycine peptide by means of a second amide bond, said conjugate of alkylenediamino linker and oligo-glycine peptide having the following formula (ii),

[symbol]

S—NH—(CH₂)ₘ—NH-(Gly)ₙ-NH₂ formula (ii)

wherein the wavy line indicates the linkage to the anthracycline derivative of formula (i), wherein m is an integer ≥1 and ≤11, preferably 2 to 5, and n is an integer ≥1 and ≤21, preferably 2 to 5. It is important to understand that, in one specific embodiment (where Streptococcus pyogenes sortase A is used, see below), the oligo-glycine (Gly)ₙ can optionally be replaced by an oligo-alanine (Ala)ₙ.

According to another group of embodiments, the oligo-glycine peptide (Gly)ₙ is, directly or by means of another linker L₁, coupled to Ring A of the anthracycline derivative of formula (ii) (where Ring A of the anthracycline core is as depicted in Shi et al., 2009). It is important to understand that, in one specific embodiment (where Streptococcus pyogenes sortase A is used, see below), the oligo-glycine (Gly)ₙ can optionally be replaced by an oligo-alanine (Ala)ₙ.

According to another group of embodiments, the oligo-glycine peptide (Glyn) is conjugated to the anthracycline derivative of formula (i) by means of an alkyleneamino linker (EA), designated as L₁, which alkyleneamino linker is conjugated to the carboxy terminus of the oligo-glycine peptide by means of an amide bond, said conjugate of alkyleneamino linker and oligo-glycine peptide having the following formula (iii)

[symbol]

-(CH₂)ₘ—NH-(Gly)ₙ-NH₂ formula (iii)

wherein the wavy line indicates the linkage to the anthracycline derivative of formula (i), wherein m is an integer ≥1 and ≤11, preferably 2 to 5, and n is an integer between ≥1 and ≤21, preferably 2 to 5. It is important to understand that, in one specific embodiment (where Streptococcus pyogenes sortase A is used, see below), the oligo-glycine (Gly)ₙ can optionally be replaced by an oligo-alanine (Ala)ₙ.

According to another group of embodiments, the linker structure L₃ comprises a peptide motif that results from specific cleavage of a sortase enzyme recognition motif.

The sortase enzyme recognition motif serves as tag for the so-called sortase-enzyme mediated antibody conjugation (SMAC-technology), which is enablingly disclosed in WO2014140317 assigned to the same applicant. The content of this publication is incorporated by reference herein. In this technology, a sortase enzyme conjugates two molecules one of which is bearing such recognition motif, while the other one is bearing a oligo-glycine peptide (Gly)ₙ. It is important to understand that, in one specific embodiment (where Streptococcus pyogenes sortase A is used, see below), the oligo-glycine (Gly)ₙ can optionally be replaced by an oligo-alanine (Ala)ₙ.

According to another group of embodiments, said sortase enzyme recognition motif comprises at least one of the following amino acid sequences: LPXTG, LPXAG, LPXSG, LAXTG, LPXTA or NPQTN, with X being any conceivable amino acid sequence.

According to another group of embodiments, the resulting linker has at least one of the following amino acid sequences: -LPXTGₙ-, -LPXAGₙ-, -LPXSGₙ-, -LAXTGₙ-, -LPXTGₙ-, -LPXTAₙ- or -NPQTGₙ-, with Gₙ being an oligo- or polyglycine with n being an integer between ≥1 and ≤21, Aₙ being an oligo- or polyalanine with n being an integer between ≥1 and ≤21, and X being any conceivable amino acid sequence.

According to another group of embodiments, the anthracycline derivative is conjugated, by means of the one or more linkers, to the carboxy terminus of the binding protein, or to the carboxy terminus of at least one domain or subunit thereof.

According to another group of embodiments, the binding protein is conjugated to the amino terminus of the oligo-glycine peptide (Gly)ₙ by means of an amide bond. It is important to understand that, in one specific embodiment (where Streptococcus pyogenes sortase A is used, see below), the oligo-glycine (Gly)ₙ can optionally be replaced by an oligo-alanine (Ala)ₙ.

The sortase which is being used is preferably a sortase A. In one embodiment, it is a sortase A from Streptococcus pyogenes or Staphylococcus aureus. In one embodiment, an engineered sortase A is being used which still recognized the sortase A recognition motif. In one embodiment, the engineered sortase A is derived from Streptococcus pyogenes sortase A.

In one other embodiment, the engineered sortase A is derived from Staphylococcus aureus sortase A.

According to another group of embodiments, the binding protein is at least one selected from the group consisting of an antibody, an antibody-based binding protein, a modified antibody format retaining target binding capacity, an antibody derivative or a fragment retaining target binding capacity, an alternative scaffold and/or an antibody mimetic.

Among the above, antibodies are the most preferred type of binding protein.

The terms “antibody”, “antibody-based binding protein”, “modified antibody format retaining target binding capacity”, “antibody derivative or fragment retaining target binding capacity” refers to polypeptide chain(s) which exhibit a strong monovalent, bivalent or polyvalent binding to a given antigen, epitope or epitopes. Antibodies, antibody-based binding proteins and antigen-binding fragments used in the invention can be generated using any suitable technology, e.g., hybridoma technology, ribosome display, phage display, gene shuffling libraries, semi-synthetic or fully synthetic libraries or combinations thereof. Antibodies, antibody-based binding proteins and antigen-binding fragments of the invention include intact antibodies and antibody fragments or antigen-binding fragments that contain the antigen-binding portions of an intact antibody and retain the capacity to bind the cognate antigen. Unless otherwise specified herein, all peptide sequences, including all antibody and antigen-binding fragment sequences are referred to in N→C order.

An intact antibody typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Each heavy chain of an antibody is comprised of a heavy chain variable region (V_{H}) and a heavy chain constant region. In the case of IgG, the heavy chain constant region is comprised of three domains, C_{H1}, C_{H2} and C_{H3}. Each light chain is comprised of a light chain variable region (V_{L}) and a light chain constant region. The light chain constant region is comprised of one domain, C_{L}. The variable regions of the heavy and light chains contain a binding domain that interacts with an antigen.

The constant regions of the antibodies may mediate the binding of the immunoglobulin to host tissues or factors, including various cells of the immune system and the first component (C1q) of the classical complement system. Monoclonal antibodies (mAbs) consist of identical antibodies molecules.

The V_{H} and V_{L} regions of an antibody can be further subdivided into regions of hypervariability, also termed complementarity-determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined, e.g., the IMGT system (Lefranc M P et al., 2015), as used herein.

Antibodies, antibody-based binding proteins and antigen-binding fragments of the invention also encompass single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment that are coded for by separate genes, a scFv has the two domains joined (e.g., via recombinant methods) by a synthetic linker. This enables them to be made as a single protein chain in which the V_{L} and V_{H} regions pair to form monovalent molecules.

Examples of antibody-based binding proteins are polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

Examples of antigen-binding fragments include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide.

Antigen-binding fragments of the present invention also encompass single domain antigen-binding units that have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The terms “human antibody” refer to an antibody, antibody-based binding protein or antigen-binding fragment that contains sequences derived from human immunoglobulins such that substantially all of the CDR regions are of human origin, and substantially all of the FR regions correspond to those of a human immunoglobulin sequence.

The terms “alternative scaffold” and “antibody mimetic” refer to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs.

Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, nucleic acid aptamers, artificial antibodies produced by molecular imprinting of polymers, peptide libraries from bacterial genomes, SH-3 domains, stradobodies, “A domains” of membrane receptors stabilised by disulfide bonds and Ca2+, CTLA4-based compounds, Fyn SH3, and aptamers (oligonucleic acid or peptide molecules that bind to a specific target molecules)

Antibodies, antibody-based binding proteins and antigen-binding fragments of the invention may bind to any suitable target on a cancer cell or within the cancer cell environment of the tumor. In one embodiment, the antibodies, antibody-based binding proteins and antigen-binding fragments of the invention bind to a target on the cancer cell surface. In a preferred embodiment, this target is exclusively expressed on the cancer cell or is overexpressed on the cancer cell relative to healthy tissue. The applicant suggests that any target to which the antibodies, antibody-based binding proteins and antigen-binding fragments of the invention bind on the cancer cell and which cause the antibodies, antibody-based binding proteins and antigen-binding fragments of the invention to be internalized into the cell, along with the anthracycline toxin moiety, resulting in immunogenic cell death of that cell and/or infiltration of immune cells (e.g., T cells) into the tumor, is a suitable target. The skilled person is capable of identifying such targets through examination of the literature and experimentation.

In one embodiment, suitable targets include ROR1, CS-1, HER2, Mesothelin and ROR2. In one embodiment, the antibodies, antibody-based binding proteins and antigen-binding fragments of the invention bind to ROR1.

According to another embodiment of the invention, the binding protein comprised in the binding protein-toxin conjugate is an antibody that
a) comprises a set of heavy chain/light chain complementarity determining regions (CDR) comprised in the heavy chain/light variable domain sequence pair set forth in the following pairs of SEQ ID NOs:
1 and 2; 3 and 4, 5 and 6 and/or 20 and 21
b) comprises a set of heavy chain/light chain complementarity determining regions (CDR) comprising the following SEQ ID NOs, in the order (HCDR1; HCDR2; HCDR3; LCDR1; LCDR2 and LCDR3)
   22, 23, 24, 25, 26, and 27,
   28, 29, 30, 31, 32, and 33,
   34, 35, 36, 37, 38, and 39, and/or
   40, 41, 42, 43, 44, and 45.
c) comprises the heavy chain/light chain complementarity determining regions (CDR) of b), with the proviso that at least one of the CDRs has up to 3 amino acid substitutions relative to the respective SEQ ID NOs, and/or
d) comprises the heavy chain/light chain complementarity determining regions (CDR) of b) or c), with the proviso that at least one of the CDRs has a sequence identity of ≥66% to the respective SEQ ID NOs, wherein the CDRs are embedded in a suitable protein framework so as to be capable to bind to ROR1 with sufficient binding affinity.

As used herein, the term “CDR” or “complementarity determining region” is intended to mean the non-contiguous antigen combining sites found within the variable region of both heavy and light chain polypeptides. These particular regions have been described by Kabat et al. (1977), Kabat et al. (1991), Chothia et al. (1987) and MacCallum et al., (1996) where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth in the table below as a comparison. Note that this numbering may differ from the CDRs that are actually disclosed in the enclosed sequence listing, because CDR definitions vary from case to case.

CDR definitions

| | Kabat | Chothia | MacCallum |
|---|---|---|---|
| VH CDR1 | 31-35 | 26-32 | 30-35 |
| VH CDR2 | 50-65 | 53-55 | 47-58 |
| VH CDR3 | 95-102 | 96-101 | 93-101 |
| VL CDR1 | 24-34 | 26-32 | 30-36 |
| VL CDR2 | 50-56 | 50-52 | 46-55 |
| VL CDR3 | 89-97 | 91-96 | 89-96 |

As used herein, the term “framework” when used in reference to an antibody variable region is entered to mean all amino acid residues outside the CDR regions within the variable region of an antibody. Therefore, a variable region framework is between about 100-120 amino acids in length but is intended to reference only those amino acids outside of the CDRs.

As used herein, the term “capable to bind to target X with sufficient binding affinity” has to be understood as meaning that respective binding domain binds the target with a K_{D} of 10⁻⁴ or smaller. K_{D} is the equilibrium dissociation constant, a ratio of k_{off}/kₒₙ, between the protein binder and its antigen. K_{D} and affinity are inversely related. The K_{D} value relates to the concentration of protein binder (the amount of protein binder needed for a particular experiment) and so the lower the K_{D} value (lower concentration) and thus the higher the affinity of the binding domain. The following table shows typical K_{D} ranges of monoclonal antibodies

K_{D} and Molar Values

| Rd value | Molar range |
|---|---|
| 10⁻⁴ to 10⁻⁶ | Micromolar (μM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

Preferably, the protein binder has up to 2 amino acid substitutions, and more preferably up to 1 amino acid substitution.

Preferably, at least one of the CDRs has a sequence identity of ≥67%; ≥68%; ≥69%; ≥70%; ≥71%; ≥72%; ≥73%; ≥74%; ≥75%; ≥76%; ≥77%; ≥78%; ≥79%; ≥80%; ≥81%; ≥82%; ≥83%; ≥84%; ≥85%; ≥86%; ≥87%; ≥88%; ≥89%; ≥90%; ≥91%; ≥92%; ≥93%; ≥94%; ≥95%; ≥96%; ≥97%; ≥98%; ≥99%, and most preferably 100% to the respective SEQ ID NO.

“Percentage of sequence identity” as used herein, is determined by comparing two optimally aligned sequences over a comparison window, wherein the portion of the polynucleotide sequence in the comparison window may comprise additions or deletions (i.e., gaps) as compared to the reference sequence (e.g., a polypeptide), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., at least 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. The disclosure provides polypeptides or polynucleotides that are substantially identical to the polypeptides or polynucleotides, respectively, exemplified herein. Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, substantial identity exists when one or two amino acid residues are conservatively substituted, according to the conservative substitutions defined herein.

Preferably, at least one of the CDRs has been subject to CDR sequence modification, including
affinity maturation
reduction of immunogenicity

Affinity maturation in the process by which the affinity of a given antibody is increased in vitro. Like the natural counterpart, in vitro affinity maturation is based on the principles of mutation and selection. It has successfully been used to optimize antibodies, antibody fragments or other peptide molecules like antibody mimetics. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range. For principles see Eylenstein et al. (2016), the content of which is incorporated herein by reference.

Engineered antibodies contain murine-sequence derived CDR regions that have been engrafted, along with any necessary framework back-mutations, into sequence-derived V regions. Hence, the CDRs themselves can cause immunogenic reactions when the humanized antibody is administered to a patient. Methods of reducing immunogenicity caused by CDRs are disclosed in Harding et al. (2010), the content of which is incorporated herein by reference.

According to another embodiment of the invention, the binding protein comprised in the binding protein-toxin conjugate is an antibody that
a) the heavy chain/light chain variable domain (HCVD/LCVD) pairs set forth in the following pairs of SEQ ID NOs:
1 and 2; 3 and 4, 5 and 6 and/or 20 and 21
b) the heavy chain/light chain variable domains (HCVD/LCVD) pairs of a), with the proviso that
   the HCVD has a sequence identity of ≥80% to the respective SEQ ID NO, and/or
   the LCVD has a sequence identity of ≥80% to the respective SEQ ID NO,
c) the heavy chain/light chain variable domains (VD) pairs of a) or b), with the proviso that at least one of the HCVD or LCVD has up to 10 amino acid substitutions relative to the respective SEQ ID NO,

said protein binder still being capable to bind ROR1 with sufficient binding affinity.

A “variable domain” when used in reference to an antibody or a heavy or light chain thereof is intended to mean the portion of an antibody which confers antigen binding onto the molecule and which is not the constant region. The term is intended to include functional fragments thereof which maintain some of all of the binding function of the whole variable region. Variable region binding fragments include, for example, functional fragments such as Fab, F(ab)₂, Fv, single chain Fv (scfv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Huston et al., (1993) or Plückthun and Skerra (1990).

Preferably, the HCVD and/or LCVD has a sequence identity of ≥81%; ≥82%; ≥83%; ≥840%; ≥85%; ≥86%; ≥870%; ≥88%; ≥89%; ≥90%; ≥91%; ≥92%; ≥93%; ≥94%; ≥95%; ≥96%; ≥97%; ≥98%; ≥99%; or most preferably 100% to the respective SEQ ID NO.

According to another embodiment of the invention, the binding protein comprises at least one amino acid substitution is a conservative amino acid substitution.

A “conservative amino acid substitution”, as used herein, has a smaller effect on antibody function than a non-conservative substitution. Although there are many ways to classify amino acids, they are often sorted into six main groups on the basis of their structure and the general chemical characteristics of their R groups.

In some embodiments, a “conservative amino acid substitution” is one in which the amino acid residue is replaced with an amino acid residue having a similar side chain. For example, families of amino acid residues having similar side chains have been defined in the art. These families include amino acids with
basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine),
nonpolar side chains (e.g., alanine, valine, leucine, isoleucine, proline, phenylalanine, methionine, tryptophan),
beta-branched side chains (e.g., threonine, valine, isoleucine) and
aromatic side chains (e.g., tyrosine, phenylalanine, tryptophan, histidine).

Other conserved amino acid substitutions can also occur across amino acid side chain families, such as when substituting an asparagine for aspartic acid in order to modify the charge of a peptide. Conservative changes can further include substitution of chemically homologous non-natural amino acids (i.e. a synthetic non-natural hydrophobic amino acid in place of leucine, a synthetic non-natural aromatic amino acid in place of tryptophan).

According to another embodiment of the invention, the binding protein has a target binding affinity to ROR1 of at least 50% compared to that of an antibody according to the above description.

As used herein the term “binding affinity” is intended to mean the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent heteromeric variable region binding fragment can exhibit altered or optimized binding affinity due to its valency.

A suitable method for measuring the affinity of a binding agent is through surface plasmon resonance (SPR). This method is based on the phenomenon which occurs when surface plasmon waves are excited at a metal/liquid interface. Light is directed at, and reflected from, the side of the surface not in contact with sample, and SPR causes a reduction in the reflected light intensity at a specific combination of angle and wavelength. Biomolecular binding events cause changes in the refractive index at the surface layer, which are detected as changes in the SPR signal. The binding event can be either binding association or disassociation between a receptor-ligand pair. The changes in refractive index can be measured essentially instantaneously and therefore allows for determination of the individual components of an affinity constant. More specifically, the method enables accurate measurements of association rates (kₒₙ) and disassociation rates (k_{off}).

Measurements of kₒₙ and k_{off} values can be advantageous because they can identify altered variable regions or optimized variable regions that are therapeutically more efficacious. For example, an altered variable region, or heteromeric binding fragment thereof, can be more efficacious because it has, for example, a higher kₒₙ valued compared to variable regions and heteromeric binding fragments that exhibit similar binding affinity. Increased efficacy is conferred because molecules with higher kₒₙ values can specifically bind and inhibit their target at a faster rate. Similarly, a molecule of the invention can be more efficacious because it exhibits a lower k_{off} value compared to molecules having similar binding affinity. Increased efficacy observed with molecules having lower k_{off} rates can be observed because, once bound, the molecules are slower to dissociate from their target. Although described with reference to the altered variable regions and optimized variable regions of the invention including, heteromeric variable region binding fragments thereof, the methods described above for measuring associating and disassociation rates are applicable to essentially any protein binder or fragment thereof for identifying more effective binders for therapeutic or diagnostic purposes.

Another suitable method for measuring the affinity of a binding agent is through surface is by FACS/scatchard analysis.

Methods for measuring the affinity, including association and disassociation rates using surface plasmon resonance are well known in the arts and can be found described in, for example, Jonsson and Malmquist, (1992) and Wu et al. (1998). Moreover, one apparatus well known in the art for measuring binding interactions is a BIAcore 2000 instrument which is commercially available through Pharmacia Biosensor, (Uppsala, Sweden).

Preferably said target binding affinity is ≥51%, ≥52%, ≥53%, ≥54%, ≥55%, ≥56%, ≥57%, ≥58%, ≥59%, ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, and most preferably ≥99% compared to that of the reference binding agent.

According to another embodiment of the invention, the binding protein competes for binding ROR1 with an antibody according to the above description.

According to another embodiment of the invention, the binding protein binds to essentially the same, or the same, region on ROR1 as an antibody according to the above description.

As used herein, the term “competes for binding” is used in reference to one of the antibodies defined by the sequences as above, meaning that the actual protein binder as an activity which binds to the same target, or target epitope or domain or subdomain, as does said sequence defined protein binder, and is a variant of the latter. The efficiency (e.g., kinetics or thermodynamics) of binding may be the same as or greater than or less than the efficiency of the latter. For example, the equilibrium binding constant for binding to the substrate may be different for the two antibodies.

Such competition for binding can be suitably measured with a competitive binding assay. Such assays are disclosed in Finco et al. 2011, the content of which is incorporated herein by reference, and their meaning for interpretation of a patent claim is disclosed in Deng et al 2018, the content of which is incorporated herein by reference.

In order to test for this characteristic, suitable epitope mapping technologies are available, including, inter alia,
X-ray co-crystallography and cryogenic electron microscopy (cryo-EM)
Array-based oligo-peptide scanning
Site-directed mutagenesis mapping
High-throughput shotgun mutagenesis epitope mapping
Hydrogen-deuterium exchange, and/or
Cross-linking-coupled mass spectrometry

These methods are, inter alia, disclosed and discussed in Banik et al (2010), and DeLisser (1999), the content of which is herein incorporated by reference.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′→3′.

### Example 1. Generation of Purified, Recombinant Anti-Human ROR1 and Isotype Control Antibodies

Expression vectors: Antibody variable region coding regions were produced by total gene synthesis (GenScript) using MNFGLRLIFLVLTLKGVQC as leader sequence, and were assembled with human IgH-γ1 and IgL-κ or IgL-λ constant regions, as applicable, in the expression vector pCB14. This vector, a derivative of the episomal mammalian expression vector pCEP4 (Invitrogen), carries the EBV replication origin, encodes the EBV nuclear antigen (EBNA-1) to permit extra-chromosomal replication, and contains a puromycin selection marker in place of the original hygromycin B resistance gene.

Expression and purification: pCB14-based expression vectors were transfected into HEK293T cells using Lipofectamine® LTX Reagent with PLUS™ Reagent (Thermo Fisher Scientific, Reinach, Switzerland, 15388100); following a 1-day incubation (37° C., 5% CO₂, growth media: Dulbecco's Modified Eagle Medium (DMEM) High Glucose (4.5 μg/L) with L-Glutamine with 10% (v/v) Fetal Calf Serum (FCS), 100 IU/mL of Pen-Strep-Fungizone and 2 mM L-glutamine (all Bioconcept, Allschwil, Switzerland)), cells were expanded under selection conditions (2 μg/mL of puromycin (Sigma-Aldrich, Buchs SG, Switzerland, P8833-25 mg stock at 2 mg/mL)). Cells were split and further expanded (37° C., 5% CO₂); once confluency was reached, tissue culture dishes were coated with 20 μg/ml poly-L-Lysine (Sigma-Aldrich, P1524) for 2 h at 37° C. and washed twice with PBS. Then, cells were trypsinized and split 1:3 onto poly-L-lysine-coated plates. Again after reaching confluency, cells were washed with PBS followed by media replacement to production media (DMEM/F-12, Gibco/Thermo Fisher Scientific, 31330-03) supplemented with 1 μg/mL puromycin (Sigma, P8833), 100 IU/mL of Pen-Strep-Fungizone (Bioconcept), 161 μg/mL of N-acetyl-L-cysteine (Sigma-Aldrich, A8199) and 10 μg/mL of L-glutathione reduced (Sigma-Aldrich, G6529). Supernatant, harvested bi-weekly and filtered (0.22 μm) to remove cells, was stored at 4° C. until purification.

For purification, filtered supernatant was loaded onto a PBS-equilibrated Protein A HiTrap column (GE Healthcare, Frankfurt am Main, Germany, 17-0405-01) or a JSR Amsphere™ Protein A column (JSR Life Sciences, Leuven, Belgium, JWT203CE) and washed with PBS; elution was performed using 0.1M glycine (pH 2.5) on an AEKTA pure (GE Healthcare). Fractions were immediately neutralized with 1M Tris-HCl buffer (pH 8.0), and analyzed for protein purity and integrity by SDS-PAGE. Protein-containing fractions were mixed and subjected to buffer exchange using Amicon filtration units (Millipore, Schaffhausen, Switzerland, UFC901008) to reach a dilution of 1:100 in PBS, and then sterile filtered using a low retention filter (0.20 m, Carl Roth, Karlsruhe, Germany, PA49.1).

Antibodies were transiently expressed in CHO cells by methods known in the art and recombinant antibodies were purified by standard protein A purification from CHO cell supernatants, as known in the art. The purity and the integrity of the recombinant antibodies were analyzed by SDS-PAGE.

TABLE 1
Anti-hROR1 and isotype control antibodies used in the Examples

| | | Antibody SEQ ID | C-Terminal Tags |
|---|---|---|---|
| Antibody | Format | HC/LC | (HC: Heavy Chain, LC: Light Chain) |
| XBR1-402 (mAb202) | IgG | HC: SEQ ID NO. 1 | HC: LPETG-Strep |
| | | LC: SEQ ID NO. 2 | LC: G₄LPETG-Strep |
| Ac10 (mAb046) | IgG | HC: SEQ ID NO. 3 | HC: LPETG-Strep |
| | | LC: SEQ ID NO. 4 | LC: G₄SLPETG-Strep |
| 2A2 (mAb066) | IgG | HC: SEQ ID NO. 5 | HC: LPETG-Strep |
| | | LC: SEQ ID NO. 6 | LC: G₄SLPETG-Strep |
| huXBR1-402-17 | IgG | HC: SEQ ID NO. 20 | HC: none |
| (mAb357) | | LC: SEQ ID NO. 21 | LC: G₄SLPETG-TwinStrep |
| Ac10 (mAb340) | IgG | HC: SEQ ID NO. 3 | HC: none |
| | | LC: SEQ ID NO. 4 | LC: G₄SLPETG-TwinStrep |

### Example 2. Conjugation of mAbs with Glycine-Modified Toxins to Form ADCs Using SMAC-Technology™

Sortase A. A recombinant and affinity purified Sortase A enzyme based on the Sortase A of Staphylococcus aureus was produced in E. coli as per the techniques described in WO2014140317A1.

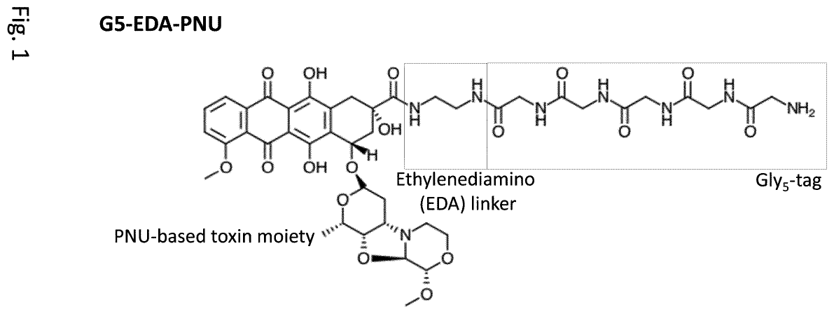

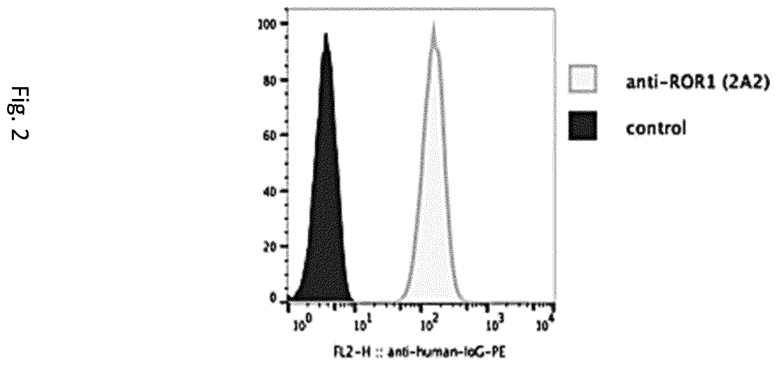

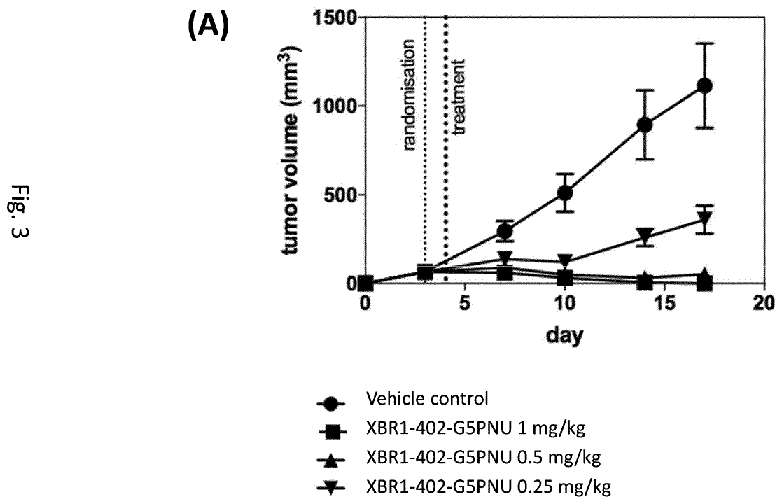

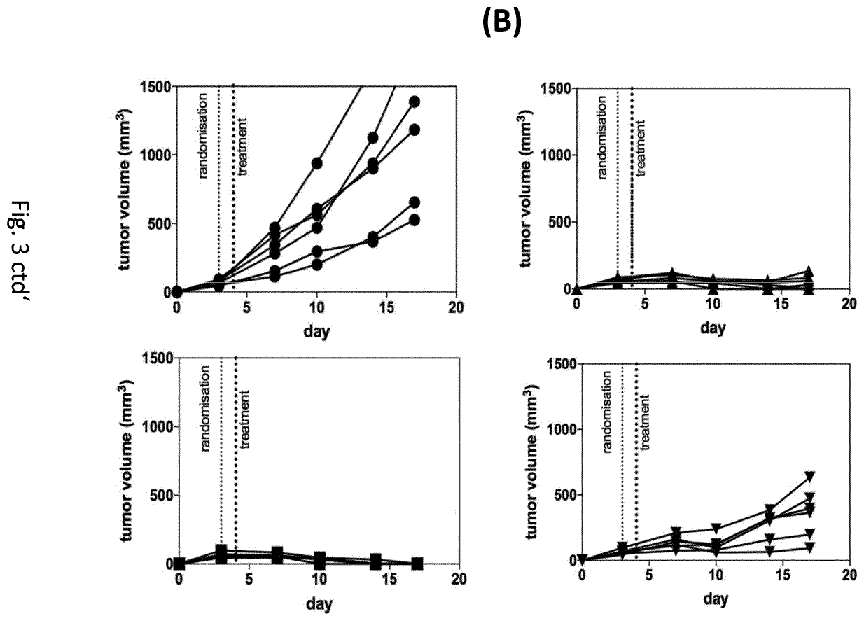

Generation of glycine-modified toxins. In order to generate SMAC-technology™ conjugated ADCs pentaglycine-EDA-PNU derivative (G5-EDA-PNU) was manufactured by Concortis (depicted in FIG. 1). The identity and purity of the pentaglycine-modified toxins was confirmed by mass-spectrometry and HPLC, respectively. The G5-EDA-PNU exhibited >95% purity, as determined by HPLC chromatography.

Sortase-mediated antibody conjugation. The above-mentioned toxin was conjugated to antibodies as per Table 2 by incubating LPETG-tagged mAbs [10 μM] with glycine modified toxin [200 μM] and 3 μM Sortase A in the listed conjugation buffer for 3.5 h at 25° C. The reaction was stopped by passing it through an rProtein A GraviTrap column (BioRad). Bound conjugate was eluted with 5 column volumes of elution buffer (0.1M glycine pH 2.5, 50 nM NaCl), with 1 column volume fractions collected into tubes containing 25% v/v 1M HEPES pH 8 to neutralise the acid. Protein containing fractions were pooled and formulated in the formulation buffer of Table 2 using a ZebaSpin desalting column.

ADC analytics. Drug-antibody ratio (DAR) was assessed by Reverse Phase Chromatography performed on a Polymer Labs PLRP 2.1 mm×5 cm, 5 m column run at 1 mL/min/80° C. with a 25-minute linear gradient between 0.05 and 0.1% TFA/H₂O and 0.04 to 0.1% TFA/CH₃CN. Samples were first reduced by incubation with DTT at pH 8.0 at 37° C. for 15 minutes. The DAR determined by Reverse Phase Chromatography is summarized in Table 2 below.

TABLE 2
Analytical summary of ADCs manufactured in this study.
DAR, drug-to-antibody ratio. ND, not determined.

| ADC | mAb (ref.) | Toxin | Conjugation Buffer | Formulation Buffer | DAR |
|---|---|---|---|---|---|
| XBR1-402- | XBR1-402 | G5-PNU | 50 mM HEPES (pH 7.5), | PBS | ND |
| G5-PNU | (mAb202) | | 150 mM NaCl, 5 mM CaCl₂ | | |
| Ac10-G5- | Ac10 | G5-PNU | 50 mM HEPES (pH 7.5), | PBS | 3.7 |
| PNU | (mAb046) | | 150 mM NaCl, 1 mM CaCl₂ | | |
| Ac10-G3- | Ac10 | G3-PNU | 250 mM HEPES, pH 7.5, | PBS | 1.9 |
| PNU | (mAb340) | | 5 mM CaCl₂, 50% Glycerol | | |
| huXBR1- | huXBR1- | G3-PNU | 250 mM HEPES, pH 7.5, | 15 mM Histidine, pH | 1.9 |
| 402-17-G3- | 402-17 | | 5 mM CaCl₂, 50% Glycerol | 6.5, 175 mM Sucrose, | |
| PNU | | | | 0.02% Tween20 | |
| huXBR1- | huXBR1- | G2-PNU | 250 mM HEPES, pH 7.5, | 15 mM Histidine pH | 2.0 |
| 402-17-G2- | 402-17 | | 5 mM CaCl₂, 50% Glycerol | 6.5, 175 mM Sucrose, | |
| PNU | | | | 0.02% Tween20 | |

From these analyses it can be concluded that the SMAC-technology™ conjugation has proceeded at high efficiency.

### Example 3. Generation of EMT-6 Cells Stably Expressing Human ROR1

Murine EMT-6 breast cancer cells were cultured in DMEM complete (Dulbecco's Modified Eagle Medium (DMEM) High Glucose (4.5 μg/L) with L-Glutamine with 10% (v/v) Fetal Calf Serum (FCS), 100 IU/mL of Pen-Strep-Fungizone and 2 mM L-glutamine (all Bioconcept, Allschwil, Switzerland)) at 37° C. and 5% CO₂. Cells were engineered to overexpress ROR1 by transposition as follows: cells were centrifuged (6 min, 290×g, 4° C.) and resuspended in RPMI-1640 media (5×10⁶ cells/mL). 400 μL of cell suspension was then added to 400 μL of RPMI containing 13.3 g of either transposable vector pPB-PGK-Puro-ROR1 (directing co-expression of full-length ROR1 (NP_005003.2) along with the puromycin-resistance gene), and 6.6 g of transposase-containing vector pCDNA3.1_hy_mPB. DNA/EMT-6 cell mixture was transferred to electroporation cuvettes (0.4 cm-gap, 165-2088, BioRad, Cressier, Switzerland) and electroporated using the Biorad Gene Pulser II with capacitance extender at 300V and 950 μF. Then, cells were incubated for 5-10 min at room temperature. Following the incubation, cells were centrifuged at 290×g for 6 min in an Eppendorf 5810R centrifuge, washed once and subsequently resuspended in DMEM complete prior to incubation at 37° C. in a humidified incubator at 5% CO₂ atmosphere. One day after electroporation, cell pools stably expressing human ROR1 were selected by adding 3 μg/mL puromycin (Sigma-Aldrich, P8833).

Single-cell clones expressing ROR1 were derived from antibiotic-selected EMT-6-ROR1 cells. Briefly, following trypsinization, 10⁶ cells were centrifuged in FACS tubes; obtained pellets were resuspended in buffer (PBS with 2% (v/v) FCS). Cells were then incubated with anti-ROR1 antibody 2A2 for 30 min (4° C., final concentration 2 μg/mL), followed by centrifugation and washing. Cells were then resuspended as previously and incubated with anti-human IgG antibody (Fc gamma-specific) PE (eBioscience, Vienna, Austria, 12-4998-82) with a 1:250 dilution in the dark (30 min, 4° C.), washed once in buffer and kept on ice until single-cell sorting of antigen-expressing cells by FACS using a FACSAriaII instrument (BD Biocsiences, San Jose, USA).

### Example 4. In Vivo Evaluation of Anti-ROR1 ADCs in EMT-6-ROR1 Syngeneic Breast Tumor Model

1×10⁶ EMT-6-ROR1 clone 14 tumor cells in 100 μl PBS were orthotopically implanted into the mammary fat pad of each BALB/c mouse. On reaching a mean tumor volume of approx. 30-80 mm³ (by caliper), on Day 0 (DO), mice were block-randomized into groups of 8 animals each according to tumour size. Mice were then treated once with different doses (0.25 mg/kg, 0.5 mg/kg and 1 mg/kg) (FIG. 3), in order to determine a sub-optimal dose, in which the ADCs only result in a partial anti-tumor response. Following this, the same models were set up for treatment with the low dose ADC, including an isotype-matched control ADC and either with or without combination treatment with 10 mg/kg anti-CTLA4 immune checkpoint inhibitor mAb 9D9 according to Table 3. Mice were monitored for up to at least 40 days, and euthanized in the case of excessive tumor burden or signs of distress. Immune checkpoint inhibitor 9D9 is an inhibitor of mouse CTLA-4 and was formulated in PBS and was purchased at BioXcell, West Lebanon, N.H., U.S.

TABLE 3
Experimental groups for in vivo evaluation of PNU toxin-
based ADCs in combination with checkpoint inhibitors

| | | Treatment | Application | | |
|---|---|---|---|---|---|
| Group | | concentration | Volume | Route | Scheme after randomization |
| 1 | Vehicle Control | — | 5 ml/kg | i.v. | Once D 0 |
| 2 | Ac10-G5-PNU (isotype control) | 0.25 mg/kg | 5 ml/kg | i.v. | Once D 0 |
| 4 | XBR1-402-G5-PNU | 0.25 mg/kg | 5 ml/kg | i.v. | Once D 0 |
| 6 | α-mCTLA-4 antibody (9D9) + | 10 mg/kg + | 5 ml/kg | i.p. + | D 2 and two further treatments 3- |
| | Ac10-G5-PNU (isotype control) | 0.25 mg/kg | | i.v. | 4 days apart + Once D 0 |
| 11 | α-mCTLA-4 antibody (9D9) + | 10 mg/kg + | 5 ml/kg + | i.p. + | D 2 and two further treatments 3- |
| | XBR1-402-G5-PNU | 0.25 mg/kg | 5 ml/kg | i.v. | 4 days apart + Once D 0 |

Tumor volumes were determined based on twice-weekly caliper measurement of tumor size. For calculation of the group median tumor volumes, the values from animals that were alive on the day in question were considered. In addition, tumor volumes of animals that were euthanized due to their tumor load were carried forward using the Last-Observation-Carried-Forward (LOCF) methodology for as long as this increased the group mean/median tumor volume.

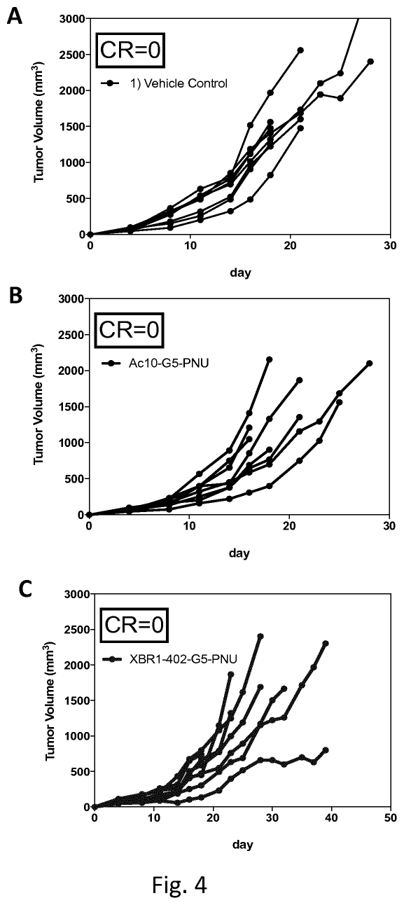

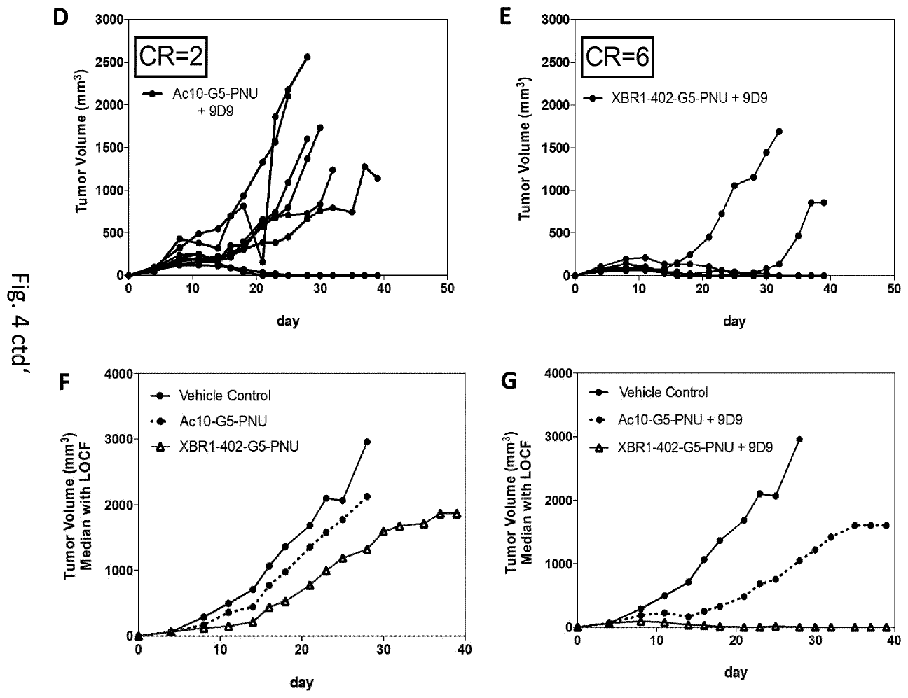

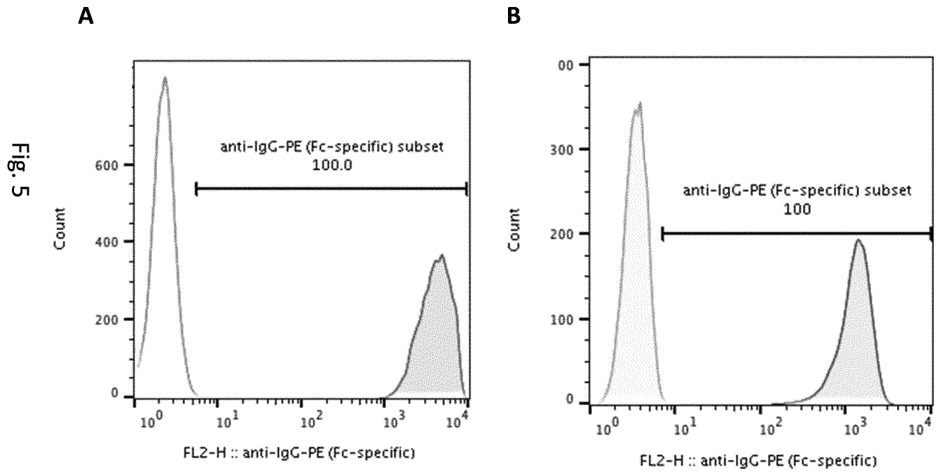

FIG. 4 presents tumor volume evolution for individual mice, as well as median tumor volume evolution. Tumor volume evolution for individual mice treated with (A) vehicle control, (B) Ac10-G5-PNU isotype control, (C) XBR1-402-G5-PNU, (D) Ac10-G5-PNU isotype control plus anti-mouse CTLA-4 antibody 9D9, (E) XBR1-402-G5-PNU plus anti-mouse CTLA-4 antibody 9D9. (F) Median group tumor volume evolution (with application of Last-Observation-Carried-Forward (LOCF) methodology) for the following groups: vehicle control, Ac10-G5-PNU isotype control and XBR1-402-G5-PNU. (G) Median group tumor volume evolution (with application of Last-Observation-Carried-Forward (LOCF) methodology) for the following groups: vehicle control, Ac10-G5-PNU isotype control and anti-mouse CTLA-4 antibody 9D9, and XBR1-402-G5-PNU and anti-mouse CTLA-4 antibody 9D9.

As shown by the results in the figure, the combination of XBR1-402-G5-PNU and anti-mouse CTLA-4 antibody 9D9 provides enhanced anti-tumor responses relative to the individual treatment with ADC at 0.25 mg/kg alone. Further, panel D shows that, relative to vehicle (Panel A) and isotype controls (Panel B), the anti-mouse CTLA-4 antibody 9D9 shows some anti-tumor efficacy, supporting that the EMT-6-ROR1 tumor model can be characterized as a hot tumor model.

Additionally, all surviving mice in group 11 (but not in other groups?—maybe then important to show?) were protected from tumor growth on re-challenge (day 102) with 1×10⁶ EMT-6-ROR1 clone 14 tumor cells, implanted in the mammary fat pad having no prior injection (not shown) indicating that only in the combination of the ADC with the checkpoint inhibitor a protective anti-tumor immune response was generated.

### Example 5. Generation of CT-26 and B16-F10 Cells Stably Expressing Human ROR1

Murine CT-26 colon carcinoma cells and B16-F10 (“B16”) melanoma cells (both from the ATCC) were cultured in RPMI media with 10% (v/v) Fetal Calf Serum (FCS), 100 IU/mL of Pen-Strep-Fungizone and 2 mM L-glutamine (all Bioconcept, Allschwil, Switzerland) at 37° C. and 5% CO₂. Cells were engineered to overexpress ROR1 by transposition as follows: cells were centrifuged in an Eppendorf 5810R centrifuge (6 min, 290×g, 4° C.) and resuspended in RPMI media (5×10⁶ cells/mL). 400 μL of cell suspension was then added to 400 μL of RPMI containing 13.3 g of transposable vector pPB-PGK-Puro-ROR1 (directing co-expression of full-length ROR1 (NP_005003.2) along with the puromycin-resistance gene), and 6.6 g of transposase-containing vector pcDNA3.1_hy_mPB. The DNA/CT-26 or B16 cell mixture was transferred to electroporation cuvettes (0.4 cm-gap, 165-2088, BioRad, Cressier, Switzerland) and electroporated using the Biorad Gene Pulser II with capacitance extender at 300V and 950 μF. Then, cells were incubated for 5-10 min at room temperature. Following the incubation, cells were centrifuged at 290×g for 6 min, washed once and subsequently resuspended in RPMI complete prior to incubation at 37° C. in a humidified incubator at 5% CO₂ atmosphere. One day after electroporation, cell pools stably expressing human ROR1 were selected by adding 3 μg/mL puromycin (Sigma-Aldrich, P8833).

Single-cell clones of CT-26 expressing ROR1 were derived from antibiotic-selected cells. Briefly, following trypsinization, 10⁶ cells were centrifuged in FACS tubes; obtained pellets were resuspended in buffer (PBS with 2% (v/v) FCS). Cells were then incubated with anti-ROR1 antibody XBR1-402 for 30 min (4° C., final concentration 2 μg/mL), followed by centrifugation and washing. Cells were then resuspended as previously and incubated with anti-human IgG antibody (Fc gamma-specific) PE (eBioscience, Vienna, Austria, 12-4998-82) at a 1:250 dilution in the dark (30 min, 4° C.), washed once in buffer and kept on ice until single-cell sorting of antigen-expressing cells by FACS using a FACSAria II instrument (BD Biocsiences, San Jose, USA). FIG. 5 (A) shows the FACS staining results of the engineered CT-26 clone 3. Expression of ROR1 on the B16 pools used in the experiment below was also determined by FACS (FIG. 5 (B)).

### Example 6. hROR1-Overexpressing CT26 Colon Carcinoma Mouse Model Treated with Anti-ROR1 ADCs and Combinations with Immune Checkpoint Inhibitors

CT26 establishes warm-to-hot tumors in mouse models, meaning that these respond to immune checkpoint inhibitors when dosed sufficiently high (Mosely, S. et al., 2017). Experimental groups, as per Table 4, each contained eight 6-8-week-old female BALB/c mice. 1×10⁶ CT-26-ROR1 clone 3 tumor cells (from Example 5) in 100 μl PBS were implanted unilaterally into the flank of each mouse. On reaching a mean tumor volume of approx. 30-100 mm³ (by caliper), on Day 0 (DO), mice were randomized into groups according to tumour size. Mice were then treated according to Table 4 and monitored three times per week for 42 days or until the tumors reached 1500 mm³ (by caliper). RPM1-14 (BioXcell, West Lebanon, N.H., U.S.) is an inhibitor of mouse PD-1 and was formulated in PBS. This immune checkpoint inhibitor was dosed below its effective dose as a monotherapy, as previously determined by Oncotest (data not shown).

TABLE 4
Experimental groups for in vivo evaluation of anti-
ROR1 ADCs with immume checkpoint inhibitors

| | | Treatment | Application | |
|---|---|---|---|---|
| Group (group size) | | concentration | Route | Scheme after randomization |
| 1 | Vehicle Control | — | i.v. | Once D 0 |
| | | | i.p. | D 0, D 4, D 8 |
| 2 | Ac10-G3-PNU (isotype) | 4 mg/kg | i.v. | Once D 0 |
| 3 | huXBR1-402-17-G3-PNU | 4 mg/kg | i.v. | Once D 0 |
| 4 | Ac10-G3-PNU + | 4 mg/kg + | i.v. + | Once D 0 + |
| | α-mPD-1 antibody (RPM1-14) | 5 mg/kg | i.p. | D 0, D 4, D 8 |
| 5 | huXBR1-402-17-G3-PNU + | 4 mg/kg + | i.v. + | Once D 0 + |
| | α-mPD-1 antibody (RPM1-14) | 5 mg/kg | i.p. | D 0, D 4, D 8 |

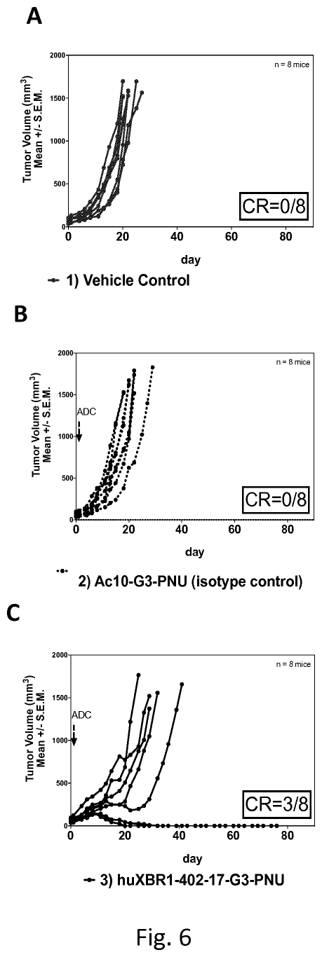

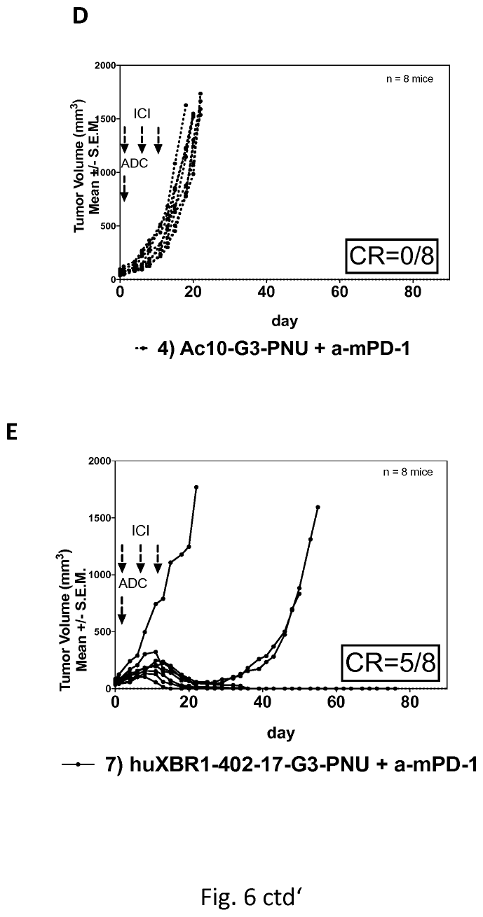

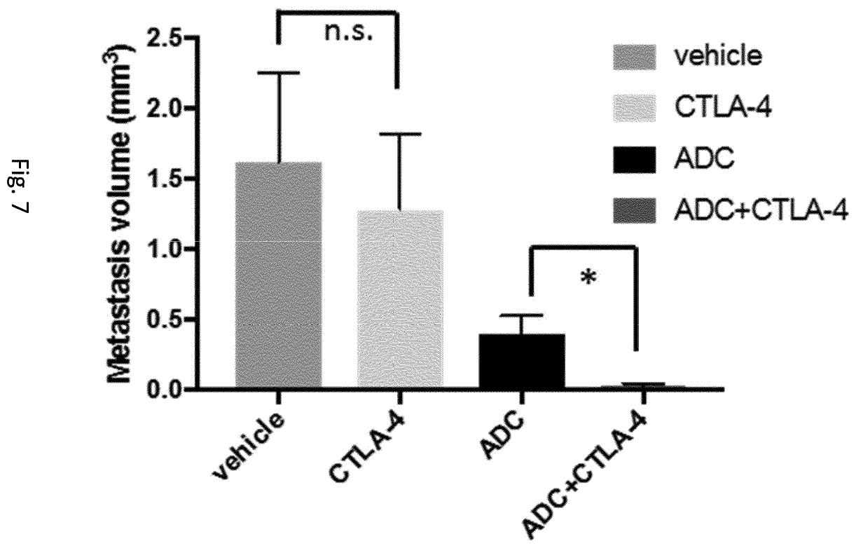

FIG. 6 presents tumor volume evolution for individual mice in each group: (A) vehicle control (untreated) of group 1, (B) isotype control of group 2, (C) anti-ROR1 ADC huXBR1-402-17-G3-PNU of group 3, (D) isotype control plus immune checkpoint inhibitor anti-mouse PD-1 antibody of group 4, (E) anti-ROR1 ADC huXBR1-402-17-G3-PNU plus immune checkpoint inhibitor anti-mouse PD-1 antibody of group 7. It is underlined that the targeted-ADC was dosed at a dose significantly lower than required for full response as monotherapy, as shown in Panel C. The results suggest a strong synergy between the evaluated immune checkpoint inhibitor and the targeted PNU derivative-comprising ADC.

### Example 7. hROR1-Overexpressing B16 Melanoma Mouse Model Treated with Anti-ROR1 ADCs and Combinations with Immune Checkpoint Inhibitors

B16 establishes poorly immunogenic (cold) tumors in mouse models (Celik, C. et al. 1983), meaning that these do not respond to immune checkpoint inhibitors when dosed within a reasonable range, i.e., up to about 50 to 100 μg/mouse (Grosso, J. et al., 2013). Experimental groups, as per Table 7, each contained ten 8-9-week-old female BL6/6NRj. 2×10⁵ B16-ROR1 tumor cells (from Example 5) in 100 μl of Hanks' Balanced Salt solution (HBSS, Thermo Fischer) were injected i.v. into each mouse at Day 0 (DO). Mice were randomized into groups by simple random allocation and treated according to Table 5, with monitoring 5 days a week over 12-19 days.

TABLE 5
Experimental groups for in vivo evaluation of anti-
ROR1 ADCs with immune checkpoint inhibitors

| | | Treatment | Application | |
|---|---|---|---|---|
| Group (group size) | | concentration | Route | Scheme after randomization |
| 1 | Vehicle Control | — | i.v. | Once D 1 |
| | | | i.p. | D 1, D 5, D 9 |
| 2 | huXBRl-402-17-G2-PNU | 4 mg/kg | i.v. | Once D 1 |
| 3 | α-mCTLA-4 antibody (9D9) | 10 mg/kg | i.p. | D 1, D 5, D 9 |
| 4 | huXBRl-402-17-G2-PNU + | 4 mg/kg + | i.v. | Once D 1 + |
| | α-mCTLA-4 antibody (9D9) | 10 mg/kg | i.p. | D 1, D 5, D 9 |

At the last day of monitoring, mice were euthanized by CO₂ followed by whole-lung collection and fixation in Bouin's solution (Labforce Bio-Optica). Experimentally induced metastasis (Saxena M. et al., 2013) on each lung surface (representing B16-ROR1 colonies) were visually counted and each was assessed for assignment to one of three bins (diameter <1 mm, 1-2 mm, >2 mm) under blinded conditions. The volume of the colonies on each lung was then determined according to the following formula (approximating each colony as a sphere):

Colony volume (mm³)=count (spots with diameter<1 mm)×0.004 mm³+count (spots with diameter 1-2 mm)×0.21 mm³+count(spots with diameter>2 mm)×0.485 mm³

Statistical analyses were performed with GraphPad Prism and unpaired T-test of average. A p-value of <0.05 was considered statistically significant (*), p-value of ≥0.5 was considered not statistically significant (n.s.).

FIG. 7 presents the colony volumes of the different treatment groups of Table 5. These results suggest, in a poorly immunogenic (“cold”) tumor with no significant effect of the immune checkpoint inhibitor, a strong synergy between the evaluated immune checkpoint inhibitor and the targeted PNU derivative-comprising ADC.

### Example 8. hROR1-Overexpressing B16 Melanoma Mouse Model Treated with Anti-ROR1 ADCs

Experimental groups, as per Table 6, contained 8-9-week-old female BL6/6NRj. 10⁶ B16-ROR1 tumor cells (from Example 5) in 100 μl of Hanks' Balanced Salt solution (HBSS, Thermo Fischer) were implanted unilaterally into the flank of each mouse. On reaching a mean tumor volume of approx. about 250 mm³ (by caliper), on Day 0 (DO), mice were randomized into groups according to tumour size as per Table 6. Mice were then treated according to Table 6 and were euthanized 24-hours after administration, followed by tumor extraction.

TABLE 6
Experimental groups for in vivo evaluation of anti-ROR1 ADC

| | | Treatment | Mice in | Administration | Treatment |
|---|---|---|---|---|---|
| Group (group size) | | concentration | group | Route | Schedule |
| 1 | Untreated control | — | 1 | — | — |
| 2 | huXBR1-402-17-G2-PNU | 4 mg/kg | 3 | i.v. | D 0 |

Tumors were snap-frozen in optimal cutting temperature compound (oct, from Fischer Scientific). Cryo-sections were stained using primary commercial anti-mouse CD4 and anti-mouse CD8 antibodies (GK1.5 BioLegend 100402, and 53-6.7 BioLegend 100702, respectively). Detection was performed using an anti-rat antibody (AlexaFluor488, Invitrogen A21208).

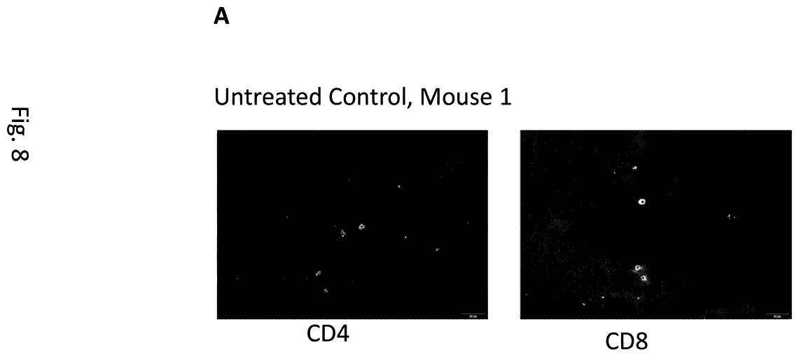

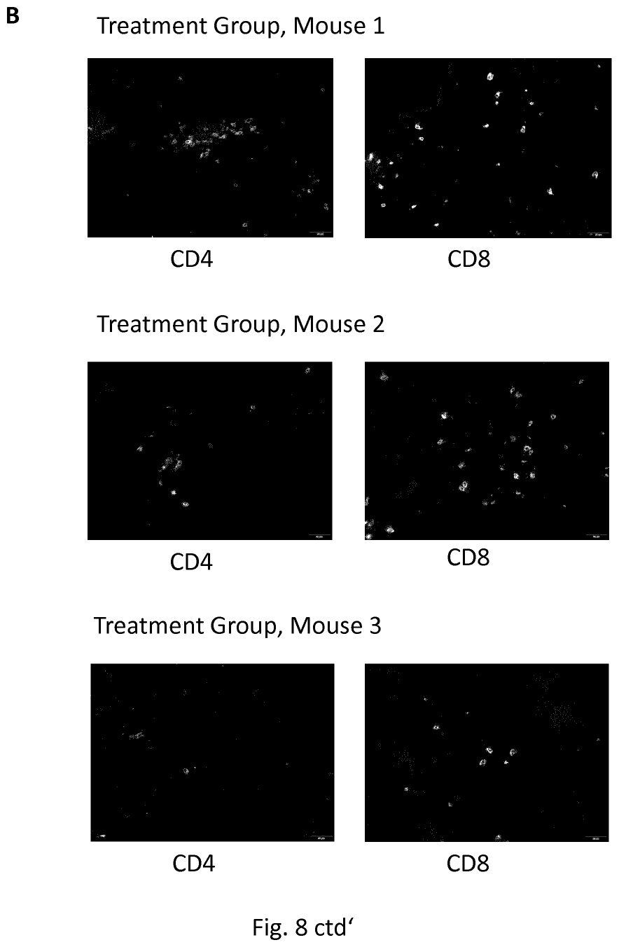

FIG. 8 shows representative images of CD4 and CD8-staining of hROR1 (human ROR1)-overexpressing B16 tumor cryo-sections from (A) the untreated control mouse, and (B) the three mice treated with ADCs (once, at 4 mg/kg) according to Table 6. As per FIG. 8(A), B16 tumors naturally contains low levels of CD4+ and CD8+ cells, in keeping with the status of B16 tumors as cold tumors. FIG. 8(B) shows that treatment with the ADC, as per the present invention, increases the number of CD4+ and CD8+ cells within the tumor.

Additionally, hROR1 (human ROR1)-overexpressing CT-26 and B-16 tumors were mechanically and enzymatically digested, and subjected to FACS staining for viable cells and immune cell markers to confirm the T-cell populations. The FACS staining scheme reported in Table 7 was used, in combination with the LIVE/DEAD™ Fixable Near-IR Dead Cell Stain Kit for 633 or 635 nm excitation staining reagent (ThermoFischer).

TABLE 7
FACS staining antibodies for immune cell population staining

| Antibody | Label | Target | clone | Cat.# | Company |
|---|---|---|---|---|---|
| anti-mouse CD45 | PE-CF592 | CD45 | 30-F11 | 562420 | BD Biosciences |
| anti-mouse CD3ε | PE/Cy7 | CD3 | 145-2C11 | 100319 | Biolegend |
| anti-mouse CD4 | PE | CD4 | RM4-4 | 116005 | Biolegend |
| anti-mouse CD8a | Alexa Fluor 488 | CD8 | 53-6.7 | 100726 | Biolegend |
| anti-mouse CD137 | APC | 4-1BB | 17B5 | 106109 | Biolegend |
| anti-mouse Ki-67 | PerCP/Cy5.5 | Ki67 | 16A8 | 652423 | ThermoFisher |

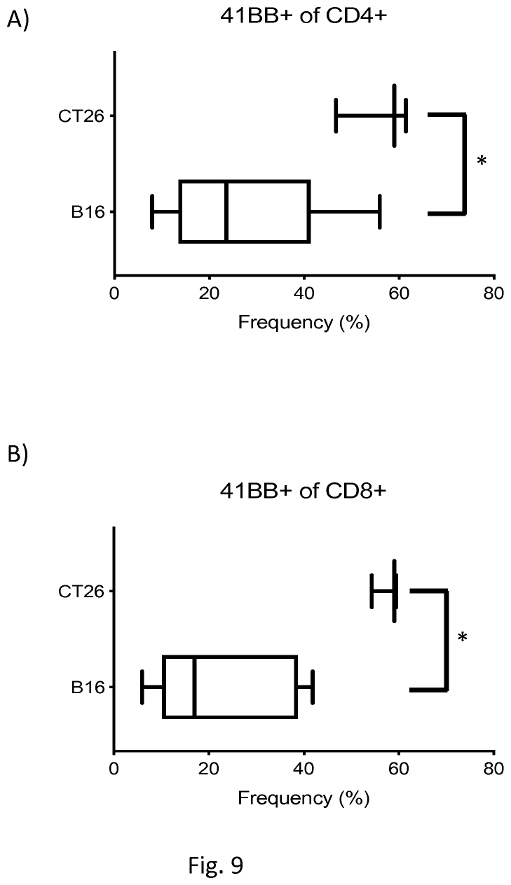

FIG. 9 shows the activation state of the CD4 (Panel A) and CD8 (Panel B) positive T-cell populations in the two tumor models (untreated), confirming the cold nature of the hROR1 (human ROR1)-overexpressing B-16 tumor model.

### REFERENCES

Galon J et al., J Pathol 2014; 232: 199-209
Pages F et al., J Clin Oncol 2009; 27: 5944-5951.
Angell H K, Galon J, Curr Opin Immunol 2013; 25: 261-267.
Galon et al. Immunity 2013; 39: 11-26.
Bu X et al., Trends in Molecular Medicine (2016), Volume 22, Issue 6, 448-451
Shi et al., Org. Biomol. Chem., 2009,7, 3709-3722
Mosely, S. et al. Cancer Immunology Research, January 2017
Saxena M and Christofori G., Molecular Oncology 7, 2013, 283-296
Celik, C. et al. Cancer Res 43, 3507-3510, 1983
Grosso, J. et al. Cancer Immunity, 22, 13(5), 2013
U. Jonsson and M. Malmquist, Adv. Biosens., 1992, 2, 291
Wu et al. Proc. Natl. Acad. Sci. USA, 95:6037-6042 (1998)
Banik, S S R; Doranz, B J (2010). “Mapping complex antibody epitopes”. Genetic Engineering & Biotechnology News. 3 (2): 25-8
DeLisser, HM (1999). Epitope mapping. Methods Mol Biol. 96. pp. 11-20
Finco et al, Comparison of competitive ligand-binding assay and bioassay formats for the measurement of neutralizing antibodies to protein therapeutics. J Pharm Biomed Anal. 2011 Jan. 25; 54(2):351-8.
Deng et al., Enhancing antibody patent protection using epitope mapping information MAbs. 2018 February-March; 10(2): 204-209
Huston et al., Cell Biophysics, 22:189-224 (1993);
Plückthun and Skerra, Meth. Enzymol., 178:497-515 (1989) and in Day, E. D., Advanced Immunochemistry, Second Ed., Wiley-Liss, Inc., New York, N.Y. (1990)
Harding, The immunogenicity of humanized and fully human antibodies. MAbs. 2010 May-June; 2(3): 256-265.
Eylenstein, et al, Molecular basis of in vitro affinity maturation and functional evolution of a neutralizing anti-human GM-CSF antibody, mAbs, 8:1, 176-186 (2016)
Kabat et al., J. Biol. Chem. 252, 6609-6616 (1977) and Kabat et al., Sequences of protein of immunological interest. (1991)
Chothia et al., J. Mol. Biol. 196:901-917 (1987)
MacCallum et al., J. Mol. Biol. 262:732-745 (1996)

### Sequences

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| | | |
|---|---|---|
| 1 | XBR1-402 HC | QEQQKESGGGLFKPTDTLTLTCTASGFDISSYYMSWVRQAPGNGLEWIGAIGISGNAYY |
| | amino acid | ASWAKSRSTITRNTNLNTVTLKMTSLTAADTATYFCARDHPTYGMDLWGPGTLVTVSSA |
| | sequence | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 2 | XBR1-402 LC | SYELTQLPSVSVSLGQTARITCEGNNIGSKAVHWYQQKPGLAPGLLIYDDDERPSGVPD |
| | amino acid | RFSGSNSGDTATLTISGAQAGDEADYYCQVWDSSAYVFGGGTQLTVTGQPKAAPSVTLF |
| | sequence | PPSSEELQANKATLVCLISDFYPGAVTVAWKADSSPVKAGVETTTPSKQSNNKYAASSY |
| | | LSLTPEQWKSHKSYSCQVTHEGSTVEKTVAPTECS |
| 3 | Ac10 HC | QIQLQQSGPEVVKPGASVKISCKASGYTFTDYYITWVKQKPGQGLEWIGWIYPGSGNTK |
| | amino acid | YNEKFKGKATLTVDTSSSTAFMQLSSLTSEDTAVYFCANYGNYWFAYWGQGTQVTVSAA |
| | sequence | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 4 | Ac10 LC | DIVLTQSPASLAVSLGQRATISCKASQSVDFDGDSYMNWYQQKPGQPPKVLIYAASNLE |
| | amino acid | SGIPARFSGSGSGTDFTLNIHPVEEEDAATYYCQQSNEDPWTFGGGTKLEIKRTVAAPS |
| | sequence | VFIFPPSDEQLKSGTASVVCLLNNFYPREAKVQWKVDNALQSGNSQESVTEQDSKDSTY |
| | | SLSSTLTLSKADYEKHKVYACEVTHQGLSSPVTKSFNRGEC |
| 5 | 2A2 HC amino | QVQLQQSGAELVRPGASVTLSCKASGYTFSDYEMHWVIQTPVHGLEWIGAIDPETGGTA |
| | acid sequence | YNQKFKGKAILTADKSSSTAYMELRSLTSEDSAVYYCTGYYDYDSFTYWGQGTLVTVSA |
| | | ASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPP |
| | | SRDELTKNQVSLTCLVKGFYPSDIAVEWESNGQPENNYKTTPPVLDSDGSFFLYSKLTV |
| | | DKSRWQQGNVFSCSVMHEALHNHYTQKSLSLSPGK |
| 6 | 2A2 LC amino | DIVMTQSQKIMSTTVGDRVSITCKASQNVDAAVAWYQQKPGQSPKLLIYSASNRYTGVP |
| | acid sequence | DRFTGSGSGTDFTLTISNMQSEDLADYFCQQYDIYPYTFGGGTKLEIKRTVAAPSVFIF |
| | | PPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |

| | | |
|---|---|---|
| 7 | Staphylococcus aureus sortase A recognition sequence 1, with X | -LPXTG |
| | being any amino acid | |
| 8 | Staphylococcus aureus sortase A recognition sequence 2, with X | -LPXAG |
| | being any amino acid | |
| 9 | recognition sequence for Staphylococcus aureus sortase A or | -LPXSG |
| | engineered sortase A 4S-9 from Staphylococcus aureus, with X | |
| | being any amino acid | |
| 10 | recognition sequence for engineered sortase A 2A-9 from | -LAXTG |
| | Staphylococcus aureus, with X being any amino acid | |
| 11 | Streptococcus pyogenes sortase A recognition sequence, with X | -LPXTA |
| | being any amino acid | |
| 12 | Staphylococcus aureus sortase recognition sequence | -NPQTN |
| 13 | Linker derived from Staphylococcus aureus sortase A recognition | -LPXT(Gn)- |
| | sequence 1, with X being any amino acid and n ≥ 1 and ≤21 | |
| 14 | Linker derived from Staphylococcus aureus sortase A recognition | -LPXA(Gn)- |
| | sequence 2, with X being any amino acid and n ≥ 1 and ≤21 | |
| 15 | Linker derived from recognition sequence for Staphylococcus | -LPXS(Gn)- |
| | aureus sortase A or engineered sortase A 4S-9 from Staphylococcus | |
| | aureus, with X being any amino acid and n ≥ 1 and ≤21 | |
| 16 | Linker derived from recognition sequence for engineered sortase | -LAXT(Gn)- |
| | A 2A-9 from Staphylococcus aureus, with X being any amino acid | |
| | and n ≥ 1 and ≤21 | |
| 17 | Linker derived from Streptococcus pyogenes sortase A recognition | -LPXT(Gn)- |
| | sequence, with X being any amino acid and n ≥ 1 and ≤21 | or |
| | | -LPXT(An)- |
| 18 | Linker derived from Staphylococcus aureus sortase recognition | -NPQT(Gn)- |
| | sequence, with n ≥ 1 and ≤21 | |
| 19 | leader sequence | MNFGLRLIFL |
| | | VLTLKGVQC |

| | | |
|---|---|---|
| 20 | huXBR1-402- | QVQLRESGPGLVKPSETLSLTCTVSGFDISSYYMSWVRQPPGKGLEWIGAIGISGNAYY |
| | 17 HC amino | ASWAKSRVTISRDTSKNQFSLKLSSVTAADTAVYYCARDHPTYGMDLWGPGTLVTVSSA |
| | acid sequence | STKGPSVFPLAPSSKSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDKTHTCPPCPAPELLG |
| | | GPSVFLFPPKPKDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQPREPQVYTLPPS |
| | | RDELTKNQVSLTCLVKGFYPSDIAVEWESNGQPENNYKTTPPVLDSDGSFFLYSKLTVD |
| | | KSRWQQGNVFSCSVMHEALHNHYTQKSLSLSPGK |
| 21 | huXBR1-402- | SYELTQPPSVSVAPGKTARITCEGNNIGSKAVHWYQQKPGQAPVLVIYDDDERPSGIPE |
| | 17 LC amino | RFSGSNSGNTATLTISRVEAGDEADYYCQVWDSSAYVFGGGTKLTVLGQPKAAPSVTLF |
| | acid sequence | PPSSEELQANKATLVCLISDFYPGAVTVAWKADSSPVKAGVETTTPSKQSNNKYAASSY |
| | | LSLTPEQWKSHRSYSCQVTHEGSTVEKTVAPTECS |
| 22 | XBR1-402 | SYYMS |
| | HCDR1 | |
| 23 | XBR1-402 | AIGISGNAYYASWAKS |
| | HCDR2 | |
| 24 | XBR1-402 | DHPTYGMDL |
| | HCDR3 | |
| 25 | XBR1-402 | EGNNIGSKAVH |
| | LCDR1 | |
| 26 | XBR1-402 | DDDERPS |
| | LCDR2 | |
| 27 | XBR1-402 | QVWDSSAYV |
| | LCDR3 | |
| 28 | Ac10 HCDR1 | YTFTDYYIT |
| 29 | Ac10 HCDR2 | WIGWIYPGSGNTKY |
| 30 | Ac10 HCDR3 | NYGNYWFAY |
| 31 | Ac10 LCDR1 | QSVDFDGDSYMN |
| 32 | Ac10 LCDR2 | VLIYAASNLES |
| 33 | Ac10 LCDR3 | QQSNEDPW |
| 34 | 2A2 HCDR1 | YTFSDYEMH |
| 35 | 2A2 HCDR2 | WIGAIDPETGGTAY |
| 36 | 2A2 HCDR3 | GYYDYDSFTY |
| 37 | 2A2 LCDR1 | QNVDAAVA |
| 38 | 2A2 LCDR2 | LLIYSASNRYT |
| 39 | 2A2 LCDR3 | QQYDIYPY |
| 40 | huXBR1-402- | FDISSYYMS |
| | 17 HCDR1 | |
| 41 | huXBR1-402- | WIGAIGISGNAYYASWA |
| | 17 HCDR2 | |
| 42 | huXBR1-402- | RDHPTYGMDL |
| | 17 HCDR3 | |
| 43 | huXBR1-402- | NIGSKAVH |
| | 17 LCDR1 | |
| 44 | huXBR1-402- | LVIYDDDERPS |
| | 17 LCDR2 | |
| 45 | huXBR1-402- | QVWDSSAY |
| | 17 LCDR3 | |

What is claimed is:

1. A binding protein-toxin conjugate comprising one or more anthracycline toxin moieties conjugated to a binding protein for use in the treatment of a patient
suffering from,
being at risk of developing, and/or
being diagnosed with
a neoplastic disease characterized as being a cold tumor.

2. A method of treating or preventing a neoplastic disease characterized as being a cold tumor, said method comprising administering to a patient a binding protein-toxin conjugate comprising one or more anthracycline toxin moieties.

3. A combination of
(i) a binding protein-toxin conjugate comprising one or more anthracycline toxin moieties, conjugated to a binding protein, and
(ii) an immune checkpoint inhibitor for use in the treatment of a patient
suffering from,
being at risk of developing, and/or
being diagnosed with
a neoplastic disease characterized as being a cold tumor,
wherein the binding protein-toxin conjugate and the immune checkpoint inhibitor are administered to the patient simultaneously or sequentially, in any order.

4. A method of treating or preventing a neoplastic disease characterized as being a cold tumor, said method comprising administering to a patient
(i) a binding protein-toxin conjugate comprising one or more anthracycline toxin moieties, conjugated to a binding protein, and
(ii) an immune checkpoint inhibitor
wherein the binding protein-toxin conjugate and the immune checkpoint inhibitor are administered to the patient simultaneously or sequentially, in any order.

5. A binding protein-toxin conjugate comprising one or more anthracycline toxin moieties, conjugated to a binding protein, for use in combination with an immune checkpoint inhibitor (for the manufacture of a medicament) for use in the treatment of a patient
suffering from,
being at risk of developing, and/or
being diagnosed with
a neoplastic disease characterized as being a cold tumor.

6. An immune checkpoint inhibitor (ICI) for use in combination with a binding protein-toxin conjugate comprising one or more anthracycline toxin moieties, conjugated to a binding protein, (for the manufacture of a medicament) for use in the treatment of a patient
suffering from,
being at risk of developing, and/or
being diagnosed with
a neoplastic disease characterized as being a cold tumor.

7. The conjugate, method, combination or ICI according to claims 1-6, wherein the neoplastic disease characterized as being a cold tumor is a tumor that is or has been refractory, resistant to immune checkpoint inhibitor treatment, or recurrent after immune checkpoint inhibitor treatment.

8. The conjugate, method, combination or ICI according to claims 1-7, wherein the neoplastic disease characterized as being a cold tumor is selected from the group consisting of
melanoma,
colorectal cancers or tumors,
pancreatic cancers or tumors
glioblastoma,
ovarian cancers or tumors, and/or
prostrate cancers or tumors.

9. The conjugate, method, combination or ICI according to claims 1-8, wherein the binding protein binds to at least one target selected from the group consisting of
ROR1
CS1
HER2
Mesothelin (MN), and/or
ROR2.

10. The conjugate, method, combination or ICI according to claims 1-9, wherein the neoplastic disease characterized as being a cold tumor characterized has having an immunoscore of <1.

11. The conjugate, method, combination or ICI according to claims 1-10, wherein at least one anthracycline toxin moiety is a derivative of the anthracycline PNU-159682 having the following formula (i) 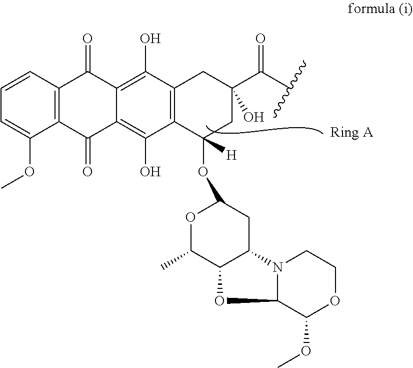
said toxin being conjugated at its wavy line to the binding protein via a linker.

12. The conjugate, method, combination or ICI according to claims 3-11, wherein the Immune Checkpoint inhibitor is at least one selected from the group consisting of
anti PD-1
anti PD-L1
anti PD-L2
anti CTLA-4
anti LAG3
anti CD40 or anti CD40L
anti TIM3,
anti OX40 or anti OX40L (CD134/CD134L),
anti CD112
anti CD155
anti B7-H3
anti B7-H4
anti IDO1
anti IDO2
anti TDO2
anti TIGIT
anti GITR, and/or
anti Galectin-9.

13. The conjugate, method, combination or ICI according to claims 1-12, wherein the conjugate comprises at its wavy line a linker structure X-L₁-L₂-L₃-Y, wherein L₁-L₃ represent linkers, and two of L₁-L₃ are mandatory, and wherein X and Y further represent each one or more optional linkers.

14. The conjugate, method, combination or ICI according to claim 13, wherein the linker structure comprises, as L₂, an oligo-glycine peptide (Gly)ₙ coupled to said anthracycline derivative, directly or by means of another linker L₁ and wherein n is an integer ≥1 and ≤21, preferably, 2 to 5.

15. The conjugate, method, combination or ICI according to claim 14, wherein the oligo-glycine peptide (Gly)ₙ is conjugated to the anthracycline derivative of formula (i) by means of an alkylenediamino linker (EDA), designated as L₁, which alkylenediamino linker is conjugated to the anthracycline derivative by means of a first amide bond, while it is conjugated to the carboxy terminus of the oligo-glycine peptide by means of a second amide bond, said conjugate of alkylenediamino linker and oligo-glycine peptide having the following formula (ii), [symbol]
S—NH—(CH₂)ₘ—NH-(Gly)ₙ-NH₂ formula (ii)
wherein the wavy line indicates the linkage to the anthracycline derivative of formula (i), wherein m is an integer ≥1 and ≤11 and n is an integer ≥1 and ≤21, preferably 2 to 5.

16. The conjugate, method, combination or ICI according to claim 14 or 15, wherein the oligo-glycine peptide (Gly)ₙ is, directly or by means of another linker L₁, coupled to Ring A of the anthracycline derivative of formula (ii).

17. The conjugate, method, combination or ICI according to claim 14, wherein the oligo-glycine peptide (Glyn) is conjugated to the anthracycline derivative of formula (ii) by means of an alkyleneamino linker (EA), designated as L₁, which alkyleneamino linker is conjugated to the carboxy terminus of the oligo-glycine peptide by means of an amide bond, said conjugate of alkyleneamino linker and oligo-glycine peptide having the following formula (iii) [symbol]
-(CH₂)ₘ—NH-(Gly)ₙ-NH₂ formula (iii)
wherein the wavy line indicates the linkage to the anthracycline derivative of formula (ii), wherein m is an integer ≥1 and ≤11 and n is an integer between ≥1 and ≤21, preferably 2 to 5.

18. The conjugate, method, combination or ICI according to claim 13-18, wherein the linker structure L₃ comprises a peptide motif that results from specific cleavage of a sortase enzyme recognition motif.

19. The conjugate, method, combination or ICI according to claim 18, wherein said sortase enzyme recognition motif comprises at least one of the following amino acid sequences: LPXTG, LPXAG, LPXSG, LAXTG, LPXTA or NPQTN, with X being any conceivable amino acid sequence.

20. The conjugate, method, combination or ICI according to claim 18 or 19, wherein the resulting linker has at least one of the following amino acid sequences: -LPXTGₙ-, -LPXAGₙ-, -LPXSGₙ-, -LAXTGₙ-, -LPXTGₙ-, -LPXTAₙ- or -NPQTGₙ-, with Gₙ being an oligo- or polyglycine with n being an integer between ≥1 and ≤21, Aₙ being an oligo- or polyalanine with n being an integer between ≥1 and ≤21, preferably 2-5, and X being any conceivable amino acid sequence.

21. The conjugate, method, combination or ICI according to any one of claims 11-20, wherein the anthracycline derivative is conjugated, by means of the one or more linkers, to the carboxy terminus of the binding protein, or to the carboxy terminus of at least one domain or subunit thereof.

22. The conjugate, method, combination or ICI according to any one of claims 14-21, wherein the binding protein is conjugated to the amino terminus of the oligo-glycine peptide (Gly)ₙ by means of an amide bond.

23. The conjugate, method, combination or ICI according to any one of claims 1-22, wherein the binding protein is at least one selected from the group consisting of an antibody, an antibody-based binding protein, a modified antibody format retaining target binding capacity, an antibody derivative or a fragment retaining target binding capacity, an alternative scaffold and/or an antibody mimetic.

24. The conjugate, method, combination or ICI according to claim 23, wherein the binding protein is an antibody that
a) comprises a set of heavy chain/light chain complementarity determining regions (CDR) comprised in the heavy chain/light variable domain sequence pair set forth in the following pairs of SEQ ID NOs: 1 and 2; 3 and 4, 5 and 6 and/or 20 and 21
b) comprises a set of heavy chain/light chain complementarity determining regions (CDR) comprising the following SEQ ID NOs, in the order (HCDR1; HCDR2; HCDR3; LCDR1; LCDR2 and LCDR3)
22, 23, 24, 25, 26, and 27,
28, 29, 30, 31, 32, and 33,
34, 35, 36, 37, 38, and 39, and/or
40, 41, 42, 43, 44, and 45,
c) comprises the heavy chain/light chain complementarity determining regions (CDR) of b), with the proviso that at least one of the CDRs has up to 3 amino acid substitutions relative to the respective SEQ ID NOs, and/or
d) comprises the heavy chain/light chain complementarity determining regions (CDR) of b) or c), with the proviso that at least one of the CDRs has a sequence identity of ≥66% to the respective SEQ ID NOs,
wherein the CDRs are embedded in a suitable protein framework so as to be capable to bind to ROR1 with sufficient binding affinity.

25. The conjugate, method, combination or ICI according to claim 23 or 24, wherein the binding protein is an antibody that comprises
a) the heavy chain/light chain variable domain (HCVD/LCVD) pairs set forth in the following pairs of SEQ ID NOs: 1 and 2; 3 and 4, 5 and 6 and/or 20 and 21
b) the heavy chain/light chain variable domains (HCVD/LCVD) pairs of a), with the proviso that
the HCVD has a sequence identity of ≥80% to the respective SEQ ID NO, and/or
the LCVD has a sequence identity of ≥80% to the respective SEQ ID NO,
c) the heavy chain/light chain variable domains (VD) pairs of a) or b), with the proviso that at least one of the HCVD or LCVD has up to 10 amino acid substitutions relative to the respective SEQ ID NO,
said protein binder still being capable to bind ROR1 with sufficient binding affinity.

26. The conjugate, method, combination or ICI according to claim 24-25, wherein the binding protein comprises at least one amino acid substitution is a conservative amino acid substitution.

27. The conjugate, method, combination or ICI according to any one of claims 1-26, wherein the binding protein has a target binding affinity to ROR1 of at least 50% compared to that of antibody according to any one of claims 24-25.

28. The conjugate, method, combination or ICI according to any one of claims 1-27, wherein the binding protein competes for binding ROR1 with an antibody according to any one of claims 24-25.

29. The conjugate, method, combination or ICI according to any one of claims 1-28, wherein the binding protein binds to essentially the same, or the same, region on ROR1 as an antibody according to any one of claims 24-25.
